(19) United States
(12) Patent Application Publication
(10) Pub. No.: US 2024/0081868 A1
(43) Pub. Date: Mar. 14, 2024

(54) MODULAR CONNECTOR ASSEMBLIES AND METHODS OF USE

(71) Applicant: Medos International Sarl, Le Locle (CH)

(72) Inventors: Joseph PETERSON, South Dartmouth, MA (US); Joseph M. DeLage, Berkley, MA (US); Ryan Thomas Andrade, Plymouth, MA (US); Paul S. Maguire, Hope Valley, RI (US); Keanan Smith, Mansfield, MA (US)

(21) Appl. No.: 18/466,192

(22) Filed: Sep. 13, 2023

Related U.S. Application Data
(60) Provisional application No. 63/406,143, filed on Sep. 13, 2022.

(51) Int. Cl. A61B 17/70; A61B 17/86

(52) U.S. Cl. CPC A61B 17/7032; A61B 17/8685; A61B 2017/00477

(57) ABSTRACT

Modular connectors and methods are disclosed that provide two sides of a connector in modular and interchangeable fashion. The modular design can be enhanced to add various useful features to one or both sides of the connectors, including the use of rods, hooks, rod receivers of various configurations, etc. This modular approach can allow the assembly of various unique connectors from a pool of components that are interchangeable while minimizing manufacturing cost and complexity.

### CROSS-REFERENCE TO RELATED APPLICATIONS

This application claims the benefit of U.S. Provisional Application No. 63/406,143, entitled “Modular Lateral Connectors,” filed on Sep. 13, 2022. The entire contents of this application are incorporated by reference herein.

### FIELD

This disclosure relates generally to surgical implants and, more particularly, to modular connector assemblies utilized in various surgical procedures, such as posterior fixation techniques utilized in spine surgery.

### BACKGROUND

In general, surgical connectors utilized in posterior fixation spinal surgeries are two-sided devices configured to couple various spinal fixation components. One such type of connector is a lateral connector, which features a first side configured to capture a rod and a second side that features a rod extending at an angle to the axis of the rod being captured. A variety of other types of connector assemblies are also possible. Manufacturing of such connectors can be challenging because forming such a connector from a single piece of stock can require a large amount of material to be removed and implant size can limit available manufacturing techniques. Furthermore, the strength of the connection between the two sides of the connector can be limited by the profile and geometric considerations.

Accordingly, there is a need for improved surgical connectors, including improved lateral and other connectors that address the shortcomings of present approaches.

### SUMMARY

Disclosed herein are modular connectors that address shortcomings in present approaches by providing the two sides of a connector in modular and interchangeable fashion. The modular design can be enhanced to add various useful features to one or both sides of the connectors. This modular approach can allow users to assemble various unique connector assemblies from a pool of components that are interchangeable.

In one aspect, a modular connector assembly can include a first connector having a first opening therein that is configured to receive a spinal fixation element, as well as a second connector extending into the first connector through a second opening formed therein. The second connector can have a bore formed in a first end thereof that is disposed within the first connector. The assembly can further include a coupler disposed within the first connector and passing through the bore of the second connector to prevent separation of the first connector and the second connector.

Any of a variety of alternative or additional features can be included and are considered within the scope of the present disclosure. For example, in some embodiments, The assembly can further include a set screw threadably coupled to the first connector. In some embodiments, the assembly can further include a spinal fixation element disposed within the first opening of the first connector, and the set screw can urge the spinal fixation element toward the coupler.

In certain embodiments, a second end of the second connector can extend through a first opening of a third connector, and a second coupler can be disposed within the third connector and passing through a bore formed in the second end of the second connector to prevent separation of the second connector and the third connector. In some embodiments, at least one of the first connector and the third connector can be configured to pivot relative to the second connector.

In certain embodiments, the first opening can be substantially U-shaped with an open proximal end and is defined by spaced apart arms of the first connector.

In some embodiments, the first opening can be formed through opposed sidewalls of the first connector with a closed proximal end.

In certain embodiments, the first connector can include an inner threaded surface configured to interface outer threads of a set screw.

In some embodiments, the second opening of the first connector can have a shape that substantially matches a shape of a portion of the second connector that extends therethrough.

In certain embodiments, the second opening of the first connector can have a shape that is larger in one dimension than a shape of a portion of the second connector that extends therethrough such that the second connector can pivot in one direction relative to the first connector. In some embodiments, the assembly can further include a spring disposed within the first connector and configured to impart a drag force on pivoting movement of the second connector relative to the first connector.

In some embodiments, the second connector can include a rod extending from the first connector. In certain embodiments, a second end of the rod can have a flange.

In certain embodiments, the second connector can include spaced apart arms defining a substantially C-shaped opening with an open end opposite the first end of the second connector that is disposed within the first connector.

In some embodiments, the second connector can include an opening formed through sidewalls thereof with a closed proximal end.

In certain embodiments, the second connector can include a hook formed opposite of the first end of the second connector that is disposed within the first connector.

In some embodiments, the second connector can include a plurality of auxiliary fixation openings formed opposite of the first end of the second connector that is disposed within the first connector.

In certain embodiments, the second connector can include a rectangular body with a plurality of bores formed therein opposite the first end of the second connector that is disposed within the first connector.

In some embodiments, the coupler can include a proximal-facing rod receiving surface. In certain embodiments, the rod receiving surface can include any of a curved surface and opposed planar surfaces that are angled toward one another.

In certain embodiments, the coupler can include a distally-extending post configured to extend through the bore formed in the second connector.

In some embodiments, the coupler can include spaced apart arms configured to interface with a retention feature on an inner surface of the first connector to prevent removal of the coupler from the first connector.

In another aspect, a method of assembling a modular connector can include inserting a first connector through an opening of a second connector to dispose a first end of the first connector within the second connector. The method can further include inserting a coupler through a second opening of the second connector and a bore formed in the first end of the first connector to prevent separation of the first connector and the second connector.

As with the instruments described above, the methods disclosed herein can include any of a variety of additional or alternative steps that are considered within the scope of the present disclosure. For example, in some embodiments, the method can further include threading a set screw into the first connector. In certain embodiments, the method can further include inserting a spinal fixation element into the first connector and rotating the set screw to urge the spinal fixation element toward the coupler.

In certain embodiments, the method can further include inserting a second end of the first connector into an opening of a third connector, as well as inserting a second coupler into the third connector and through a bore formed in the second end of the first connector to prevent separation of the first connector and the third connector. In some embodiments, the method can include pivoting at least one of the second connector and the third connector relative to the first connector.

In some embodiments, the method can further include inserting a spring into the second connector to impart a drag force on pivoting movement of the first connector relative to the second connector. In certain embodiments, the method can further include inserting the coupler through a bore formed in the spring.

In certain embodiments, the method can further include pivoting the first connector relative to the second connector.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1A is a front perspective view of one embodiment of a modular assembly according to the present disclosure;

FIG. 1B is a rear perspective view of the modular assembly of FIG. 1A;

FIG. 2 is a perspective view of one embodiment of a connector rod;

FIG. 3 is a perspective view of one embodiment of a connector body;

FIG. 4 is a perspective view of one embodiment of a rod seat;

FIG. 5 is an exploded perspective view of one embodiment of a modular assembly according to the present disclosure showing an order of assembly;

FIG. 6A is a top view of one embodiment of a modular assembly according to the present disclosure;

FIG. 6B is a cross-sectional side view of the modular assembly of FIG. 6A;

FIG. 6C is a front view of the modular assembly of FIG. 6A;

FIG. 7A is a side view of one embodiment of a connector body;

FIG. 7B is a front view of the connector body of FIG. 7A;

FIG. 7C is a top view of the connector body of FIG. 7A;

FIG. 7D is a cross-sectional top view of the connector body of FIG. 7A taken along the line 705 of FIG. 7B;

FIG. 8 is a top view of one embodiment of a connector rod;

FIG. 9A is a top view of one embodiment of a modular assembly according to the present disclosure;

FIG. 9B is a side view of the modular assembly of FIG. 9A;

FIG. 9C is a front view of the modular assembly of FIG. 9A;

FIG. 10A is a cross-sectional side view of one embodiment of a modular assembly according to the present disclosure;

FIG. 10B is a side view of the modular assembly of FIG. 10A;

FIG. 11A is a side view of one embodiment of a connector body;

FIG. 11B is a front view of the connector body of FIG. 11A;

FIG. 11C is a top view of the connector body of FIG. 11A;

FIG. 11D is a cross-sectional top view of the connector body of FIG. 11A taken along the line 1105 of FIG. 11B;

FIG. 12 is a top view of one embodiment of a connector rod;

FIG. 13A is a top view of one embodiment of a modular assembly according to the present disclosure;

FIG. 13B is a side view of the modular assembly of FIG. 13A;

FIG. 13C is a front view of the modular assembly of FIG. 13A;

FIG. 14A is a side view of one embodiment of a modular assembly according to the present disclosure;

FIG. 14B is a cross-sectional side view of the modular assembly of FIG. 14A;

FIG. 15A is a perspective view of one embodiment of a modular assembly according to the present disclosure;

FIG. 15B is an exploded perspective view of the modular assembly of FIG. 15A;

FIG. 16A is a cross-sectional detailed view of one embodiment of a rod seat partially inserted into a connector body;

FIG. 16B is a cross-sectional detailed view of the rod seat fully inserted into the connector body of FIG. 16A;

FIG. 17A is a top view of one embodiment of a modular assembly according to the present disclosure;

FIG. 17B is an alternative top view of the modular assembly of FIG. 17A;

FIG. 17C is a perspective view of the modular assembly of FIG. 17A;

FIG. 17D is a top view of a connector rod of the modular assembly of FIG. 17A;

FIG. 17E is a cross-sectional side view of the modular assembly of FIG. 17A;

FIG. 18A is a perspective view of one embodiment of a modular assembly according to the present disclosure having a connector body and a hook in a first orientation;

FIG. 18B is a perspective view of the modular assembly of FIG. 18A with the hook in a second orientation;

FIG. 18C is a perspective view of the modular assembly of FIG. 18A with the hook in a third orientation;

FIG. 18D is a perspective view of the modular assembly of FIG. 18A with the hook in a fourth orientation;

FIG. 19A is a perspective view of one embodiment of a modular assembly according to the present disclosure having a connector body and a multipoint fixation component;

FIG. 19B is an alternative perspective view of the modular assembly of FIG. 19A;

FIG. 19C is a perspective view of the modular assembly of FIG. 19A where the multipoint fixation adapter includes an extended connecting rod portion;

FIG. 19D is an alternative perspective view of the modular assembly of FIG. 19C;

FIG. 20A is a top view of one embodiment of a modular assembly according to the present disclosure having a first connector body and a second, in-line connector;

FIG. 20B is a perspective view of the modular assembly of FIG. 20A;

FIG. 20C is another perspective view of the modular assembly of FIG. 20A;

FIG. 21 is a side view of one embodiment of a modular assembly according to the present disclosure having a first connector body and a second, C-shaped connector body;

FIG. 22A is a perspective view of the modular assembly of FIG. 21;

FIG. 22B is a perspective view of the C-shaped connector body of FIG. 21;

FIG. 23 is a side view of one embodiment of a modular assembly according to the present disclosure having a first connector body and a second, O-shaped connector body;

FIG. 24A is a perspective view of the modular assembly of FIG. 23;

FIG. 24B is a perspective view of the O-shaped connector body of FIG. 23;

FIG. 25A is a side view of one embodiment of a modular assembly according to the present disclosure;

FIG. 25B is a perspective view of the modular assembly of FIG. 25A;

FIG. 26A is a perspective view of one embodiment of a modular connector assembly according to the present disclosure;

FIG. 26B is a side view of the modular assembly of FIG. 26A;

FIG. 26C is a bottom view of the modular assembly of FIG. 26A;

FIG. 27A is a top view of the modular assembly of FIG. 26A coupled to a spinal rod;

FIG. 27B is a side view of the modular assembly of FIG. 26A coupled to a spinal rod;

FIG. 28 is a top view of one embodiment of a posterior fixation construct utilizing a modular connector assembly;

FIG. 29 is a top view of one embodiment of a posterior fixation construct utilizing a modular connector assembly;

FIG. 30 is a perspective view of one embodiment of a modular assembly according to the present disclosure having a flanged connector rod;

FIG. 31A is a side view of the modular assembly of FIG. 30;

FIG. 31B is a bottom view of the modular assembly of FIG. 30;

FIG. 32 is a top view of the modular assembly of FIG. 30 coupled to a spinal rod;

FIG. 33 is a side view of the modular assembly of FIG. 30 coupled to a spinal rod;

FIG. 34 is a perspective view of one embodiment of a posterior fixation construct utilizing a modular connector assembly; and

FIG. 35 is a top view of one embodiment of a posterior fixation construct utilizing a modular connector assembly.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure. Additionally, to the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used.

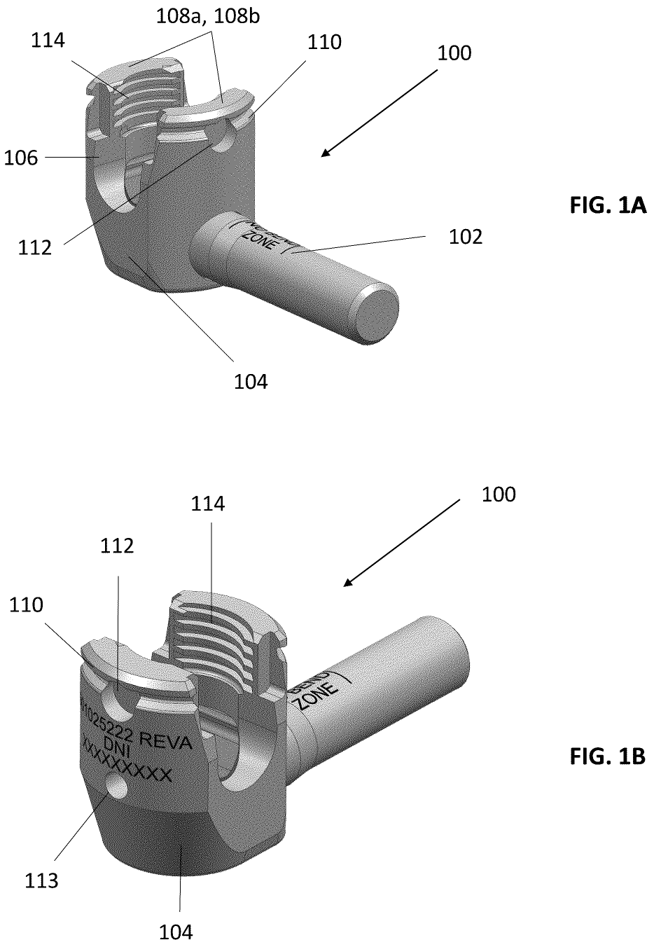

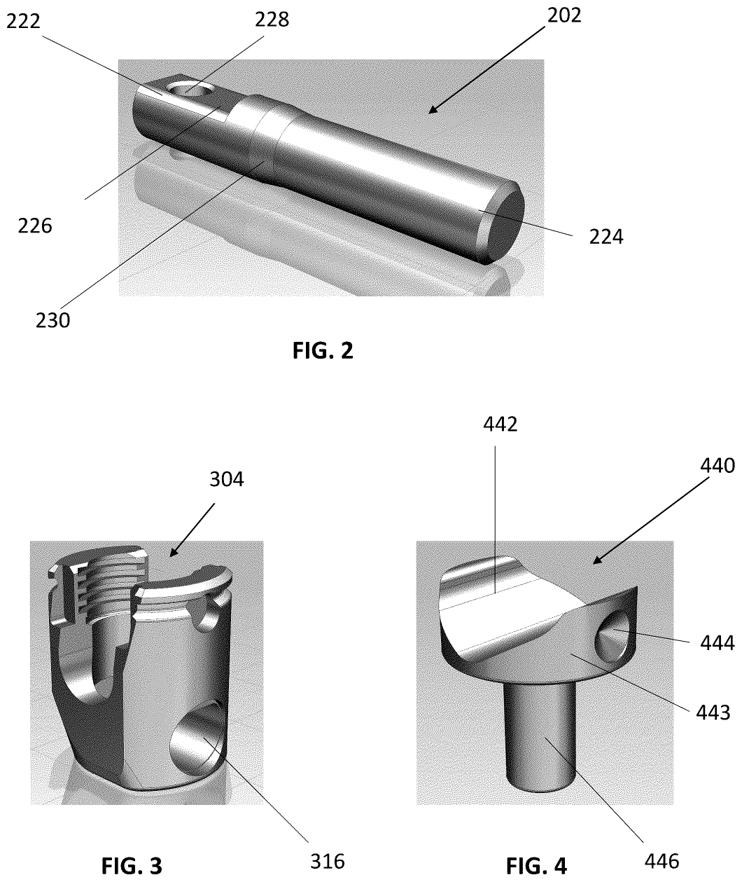

FIGS. 1A-1B illustrate one embodiment of a modular connector assembly utilizing a statically-positioned connector rod extending from one side of a connector body that is configured to capture a spinal fixation element within a U-shaped recess thereof. In FIG. 1A, the modular connector assembly 100 can include a connector body 104 and a connector rod 102 having a first end (not shown in FIG. 1A, see FIG. 2) capable of coupling to the connector body 104. The connector body 104 can have a proximal portion defined by a pair of spaced apart arms 108a, 108b which form a U-shaped recess or opening 106 (also referred to as a rod-receiving recess) therebetweeen to receive a spinal fixation element (not shown), such as a spinal rod. An internal surface of the spaced apart arms 108a, 108b can include a threaded surface 114 that can engage with a set screw or other locking element received therebetween to lock a spinal rod within the connector body 104. As discussed in detail below, the connector body 104 can have multiple distinct engagement or attachment features to facilitate coupling of the connector body 104 to surgical instruments during use.

For example, the proximal end of the connector body 104 can include a groove or channel 110 in an outer surface of the proximal end of the connector body 104 (i.e., on each of the spaced apart arms 108a, 108b). This groove 110 can define a “top-notch” feature that can be engaged with a corresponding portion of an instrument, such as a projection, to facilitate coupling of the instrument to the connector body 104. The connector body 104 can also include a proximal rocker feature or recess 112 formed in the connector body 104 that can facilitate the reduction of a rod or other spinal fixation element distally into the U-shaped recess 106 of the connector body 104. The proximal rocker feature 112 can allow a rocker instrument to pivotably couple to the connector body 104 for reduction of a spinal fixation element into the connector body 104 via a levering or rocking motion. The proximal rocker feature 112 can be a bilateral circular depression or recess that intersects the top-notch feature 110. Additionally or alternatively, the connector body 104 can include a more distal recess 113 (in FIG. 1B) formed in the connector body 104 by a swaging operation used to retain a rod seat within the connector body 104. In some embodiments, this more distal recess can also be utilized as a rocker feature to facilitate reduction of a spinal fixation element into the U-shaped recess 106 of the connector body 104. Additional disclosures of rocker and other features that can be included in the connector body 104, which can be similar to a polyaxial screw receiver head, are provided in U.S. Patent Publication No. 2022/0280200, entitled “Multi-feature Polyaxial Screw,” the entire contents of which are incorporated herein by reference.

FIGS. 2-4 illustrate individual modular components of a connector assembly, like the one shown in FIG. 1A, including a rod in FIG. 2, a connector body in FIG. 3, and a rod seat in FIG. 4. FIG. 2 is a perspective view of one embodiment of a connector rod 202 of the present disclosure. The connector rod 202 can include a first end 222 configured for coupling to a socket of a connector body, and a second end 224 that can be coupled to other surgical components or instruments, e.g., can be received within a rod-receiving recess of a polyaxial bone screw, etc. The first end 222 can have a substantially cylindrical shape with a flat surface 226 for securely coupling the connector rod 202 to the connector body 304 via a complementary-shaped socket. The first end 222 can also include a slot or bore 228 for receiving a rod seat 440 within the connector body 304, thereby securing the components together.

The connector rod 202 can include a taper 230 between the first end 222 and the second end 224 of the connector rod 202. In some embodiments, the first end 222 can include opposing flat surfaces for coupling to the connector body. Thus, in some embodiments, the first end 222 may have a diameter greater than the diameter of the second end 224.

FIG. 3 illustrates one embodiment of a connector body 304 having an opening or socket 316 configured to couple to the connector rod 202 of FIG. 2. The opening 316 can have a shape complementary to the first end 222 of the connector rod 202. The connector body 304 of FIG. 3 can also include any of the features described herein, such as those discussed above with regard to the connector body 104 of FIG. 1A.

FIG. 4 is a perspective view of one embodiment of a rod seat 440. The rod seat 440 can include a proximal end 442 having a substantially U-, V-, or other similarly shaped surface that can seat a spinal rod. For example, in some embodiments, the proximal surface 442 can have a U-Shaped surface with a radius of curvature that matches that of a spinal fixation rod to be received thereon. In some embodiments, the surface can have opposed planar or flat surfaces that are angled relative to one another, which can facilitate interfacing with spinal fixation rods of various diameters. On an external surface 443 of the proximal end 442, the rod seat 440 can include a depression or indent 444 for locking the rod seat 440 into a connector body via a swaging operation. The rod seat can include a distal post 446 configured to pass through the bore or slot 228 of the connector rod 202. For example, when the rod seat 440 is inserted into the connector body 304 having a connector rod 202 inserted into the socket 316 of the connector body, the distal post 446 can couple the components together by passing through the slot 228 of the connector rod 202 and then being locked into place within the connector body 304 by a swaging operation. The rod seat 440 can be locked into the connector body in a number of ways, including the swage lock described here or a press-fit lock, as described below.

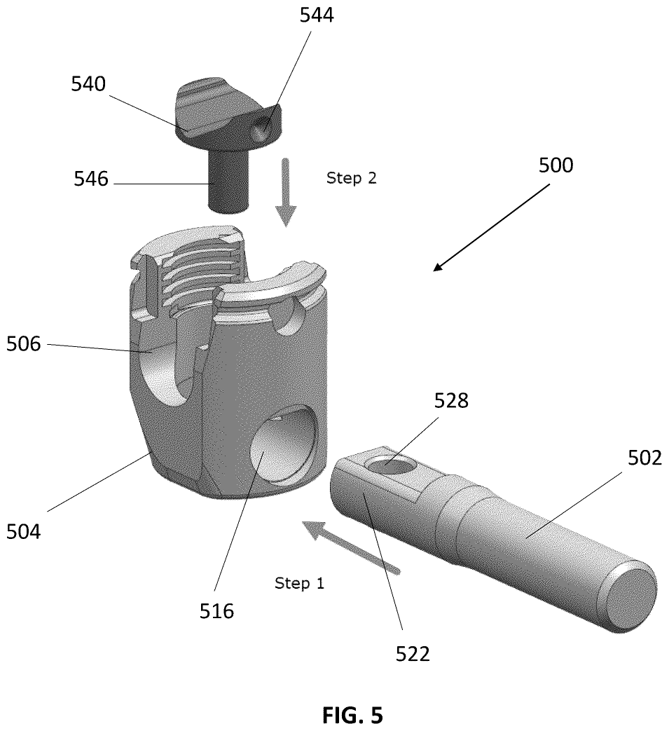

FIG. 5 illustrates an assembly process for one embodiment of a modular assembly similar to the one shown in FIG. 1A. First, a connector rod 502 can be laterally inserted into a socket 516 formed in a connector body 504. In some embodiments, the connector rod 502 and the socket 516 can be sized to provide a press fit or at least some amount of provisional holding when the connector rod 502 is inserted into the socket 516. After inserting the connector rod 502 into the socket 516 of the connector body 504, the rod seat 540 can be advanced distally into the U-shaped opening 506 of the connector body 504 from a proximal end thereof. The distally extending post 546 on the rod seat 540 can pass through a bore 528 formed in the first end 522 of the connector rod 502 to prevent separation of the connector rod 502 from the connector body 504. In some embodiments, the rod seat 540 can then be locked into place within the U-shaped opening 506 of the connector body 504 with a swaging operation or a press fit connection.

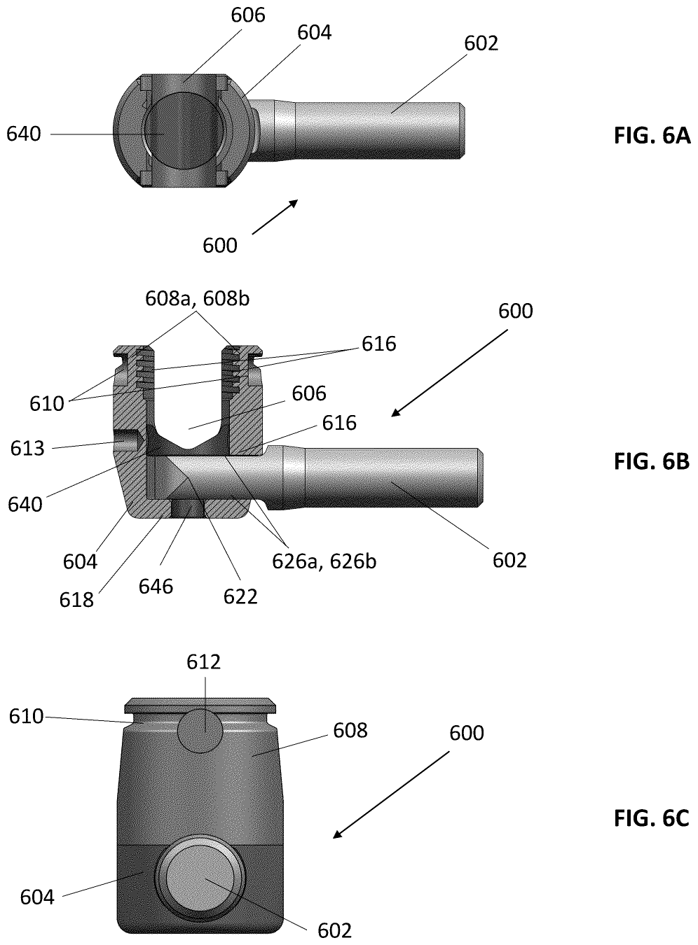

FIGS. 6A-6C illustrate another embodiment of a modular connector assembly including a connector body and a statically positioned connector rod extending from a socket formed in the connector body. In FIG. 6A, a top view is shown of a modular connector assembly 600 where a rod seat 640 is positioned within the connector body 604. The connector rod 602 extends perpendicularly from a central longitudinal axis of the rod seat 640. In FIG. 6B, a cross-sectional side view can be seen of the modular assembly 600 of FIG. 6A. The connector body 604 can include a pair of spaced apart arms 608a, 608b at the proximal end of the connector body 604 that form a U-shaped recess 606. The internal surface of each spaced apart arm 608a, 608b includes threading 616 for coupling to a set screw. The outer surface of each spaced apart arm 608a, 608b can include a top-notch feature 610, as well as various rocker features, for engaging various instruments. The connector body 604 can also include an end slot 618 on the distal end of the connector body 604. The end slot 618 can receive the distal post 646 of the rod seat 640 and help strengthen the connection between the connector rod 602, connector body 604, and rod seat. In certain embodiments, the distal post 646 of the rod seat 640 has a length that allows the terminal end of the distal post 646 to be flush with the bottom of the connector body 604 when the connector body 604 and the rod seat 640 are coupled.

The connector body 604 can receive the connector rod 602 through a distal socket 616. The first end 622 of the connector rod 602 can include opposing flat surfaces 626a, 626b for coupling to the socket 616 of the connector body 604, and the socket 616 can have corresponding surfaces such that the interface between the connector body 604 and the rod 602 resists relative movement therebetween.

FIG. 6C shows a front view of the connector system of FIG. 6A. In this view, the connector rod 602 extends from the connector body 604 at an angle perpendicular to a central longitudinal axis of the connector body 604. The external surface of the proximal end 608 of the connector body 604 (i.e., the external surface of one of the pair of spaced apart arms) can include a top-notch feature 610 and a proximal rocker feature 612 for coupling various instruments to the connector body 604. As noted above, a variety of other features can be incorporated into the connector body 604 as well, such as various tapers or other shapes, features to facilitate engagement with instruments via one or both of the spaced apart arms, etc. Additional disclosure regarding such features can be found in the above-noted U.S. Patent Publication No. 2022/0280200 that is incorporated by reference herein.

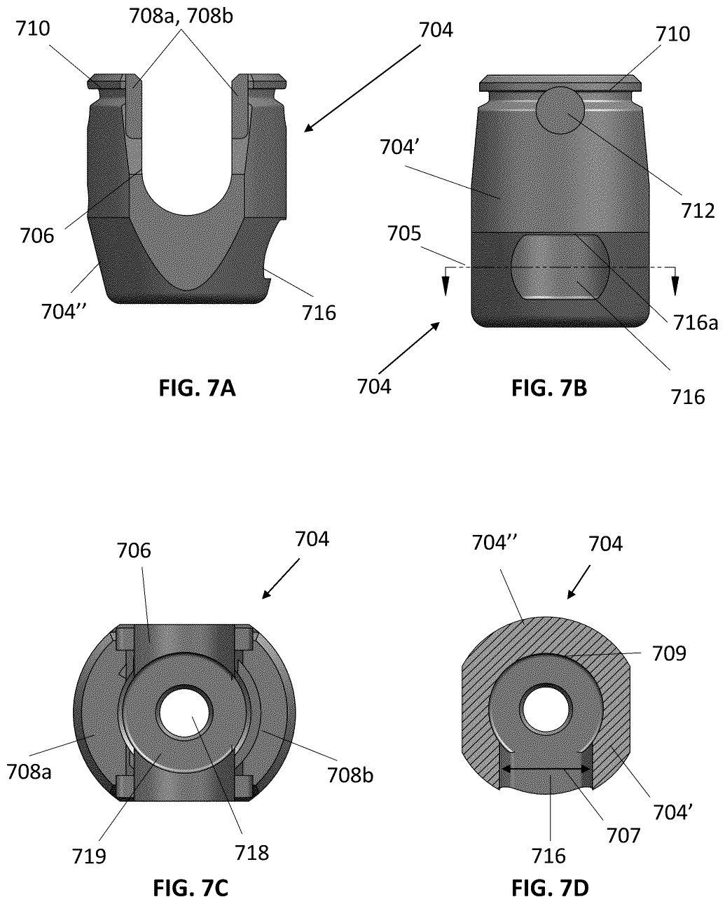

FIGS. 7A-7D illustrate one embodiment of a connector body similar to the one shown in FIG. 6A in greater detail. In FIG. 7A, the connector body 704 includes a pair of spaced apart arms 708a, 708b at the proximal end of the connector body 704 that form a U-shaped recess 706. The outer surface of each spaced apart arm 708a, 708b includes a top-notch feature 710 for engaging various instruments. Near the distal end of the connector body 704 is a socket 716 for receiving a connector rod. Notably, the socket 716 for receiving the connector rod does not extend through to the opposing outer surface 704″ of the connector body 704. In FIG. 7B, the front side 704′ of the connector body 704 of FIG. 7A is shown, including the socket 716. As noted, the proximal end of the connector body 704 can include a top notch feature 710. Additionally or alternatively, the proximal end can include a proximal rocker feature 712. The socket 716 can have a shape that complements the cross-sectional shape of a connector rod to be used therewith. In some embodiments, the socket 716 can be substantially round or oval in shape. In some embodiments, the socket 716 can include at least one flat surface 716a that corresponds to a flat surface on a first end of a connector rod. Inclusion of non-circular features, such as the flat surface 716a, can ensure proper assembly of the connector body to the connector rod (i.e., the rod can only be inserted in a desired orientation relative to the body) and can aid in resisting relative movement between the rod and body once assembled.

FIG. 7C depicts a top view of the connector body 704 of FIG. 7A. From this view, the pair of spaced apart arms 708a, 708b form a recess 706. An end slot 718 can be seen at the distal end 719 of the connector body 704. FIG. 7D shows a cross sectional view taken from the dotted line 705 of FIG. 7B. The distal socket 716 does not extend through the opposing side 704″ of the connector body 704, showing a stop for insertion of the first end of the connector rod within the connector body 704. Further, a width 707 of the socket 716 can closely conform to a diameter or width of the rod to be used therewith, such that the rod is not able to pivot left or right in the view of FIG. 7D once inserted into the socket 716. These features can help maintain the connector rod in a desired orientation relative to the connector body 704 after assembly. Further, at least the socket width 707 can be in contrast to embodiments described below that provide for some rod movement after assembly, e.g., pivoting about a central axis of the connector body.

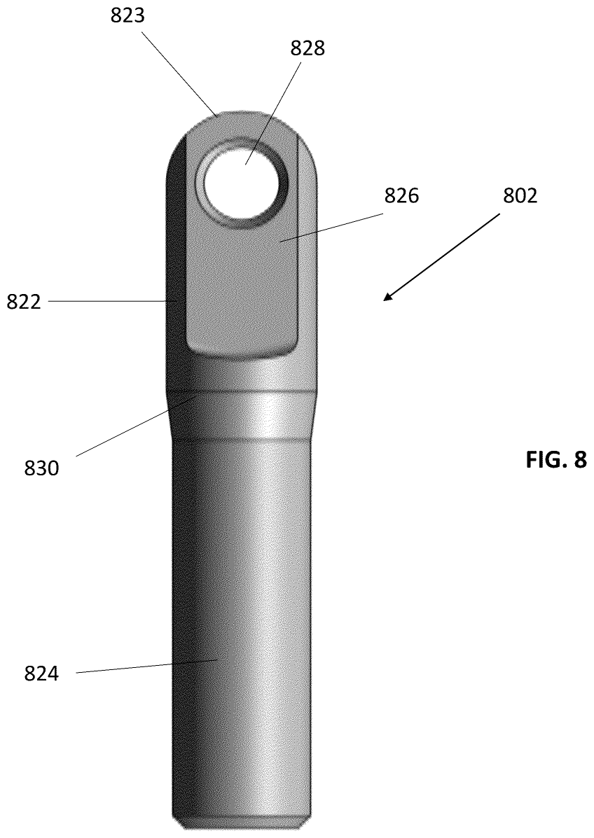

FIG. 8 illustrates one embodiment of a connector rod 802 that is similar to the rod of FIG. 6A in greater detail. Its outer shape can be configured to match with the contour of a socket formed in the connector body, as shown in FIGS. 7B and 7D, in order to resist relative movement between the connector rod and the connector body once coupled by inserting the connector rod into the socket of the connector body. As shown, the connector rod 802 can include a first end 822 and a second end 824. The first end 822 can have at least one flat surface 826 for matching the socket of the connector body. The first end 822 can also include a bore or slot 828 for receiving a distal post of a rod seat. In some embodiments, the first end 822 of the connector rod can include a rounded terminus 823. The rounded terminus 823 can match a shape of an internal surface of the connector body (e.g., curve 709 shown in FIG. 7D). The connector rod 802 can also include a taper 830 that allows the first end 822 and the second end 824 of the connector rod 802 to have different diameters. The second end 824 of the connector rod 802 can be substantially cylindrical, though other shapes are also possible, and can be utilized for coupling to additional or alternative instruments or tools. For example, in some embodiments a posterior fixation construct can include capturing the second end 824 of the connector rod 802 within a receiver head of a polyaxial bone screw implanted in a patient.

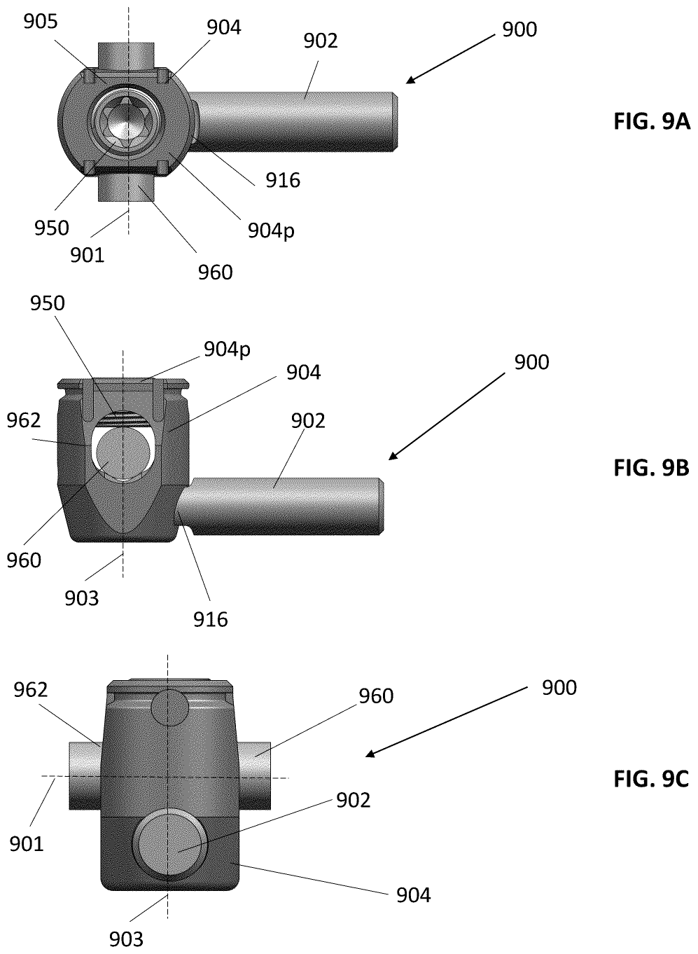

FIGS. 9A-9C illustrate one example of an alternative modular connector body type that can be utilized. In particular, the illustrated connector body is a closed-top body 904 that can be referred to as an “O” type connector body, in contrast to the above-described embodiments with a U-shaped recess open at its proximal end that can be referred to as “U” type connectors. As such, the modular assemblies shown in FIGS. 9A-9C can be referred to as “O-R” assemblies (i.e., an “O” type connector body on one side of the assembly and a connecting rod on the other), whereas the above-described embodiments can be referred to as “U-R” assemblies (i.e., a “U” type connector body on one side of the assembly and a connecting rod on the other). The closed-top connector body 904 can include a rod capture bore 962 formed along a horizontal or medial-lateral axis 901 of the closed-top connector body 904 (i.e., perpendicular to a longitudinal or proximal-distal axis 903 of the connector body). The connector body 904 can also include a set screw bore 905 formed in the proximal end 904p, or closed top, of the connector body 904 for coupling a set screw 950 to the closed-top connector body 904. The closed-top connector body 904 can include a socket 916 for receiving a statically-positioned connector rod 902 extending perpendicular to an axis of the rod capture bore 962 formed in the body of the closed-top connector 904. The closed-top connector body 904 can capture a spinal rod 960 by sliding the spinal rod 960 through the rod capture bore 962 and securing the spinal rod 960 with a set screw 950, rather than top-loading into a U-shaped rod seat, as is possible with the above-described embodiments.

FIG. 9A depicts a top view of a modular connector assembly utilizing a closed-top connector body 904. The closed-top connector body 904 can include a proximal end 904p that is substantially planar. A set screw bore 905 in the proximal end 904p can allow a set screw 950 to couple to the closed-top connector body 904. A spinal rod 960 can extend through a rod capture bore in the closed-top connector body 904. A connector rod 902 can couple to the closed-top connector body 904 via a socket 916. FIG. 9B shows a side view of the connector system of FIG. 9A. The spinal rod 960 can extend through a rod capture bore 962 that follows a horizontal axis 901 through the closed-top connector body 904. The set screw 950 can be coupled to the closed-top connector body 904 through the proximal end 904p of the closed-top connector body 904, thereby securing the spinal rod 960 in the rod capture bore 962. The socket 916 in the closed-top connector body 904 can accept a connector rod 902 such that the rod extends perpendicular to the horizontal axis 901 of the rod capture bore 962. In some embodiments, however, the socket 916 can be rotated about the axis 903 by 90 degrees, or any other angle, such that connector rod 902 can extend parallel to the rod 960, or at any desired angle relative thereto. Such a modification is possible with regard to any of the various embodiments disclosed herein. FIG. 9C further illustrates the perpendicular axes 901, 903 of the rod capture bore 962 and the connector rod 902. The connector body 904 of FIGS. 9A-9C can also include any of the features described herein, such as those described above with regard to the connector body of FIG. 1A, etc.

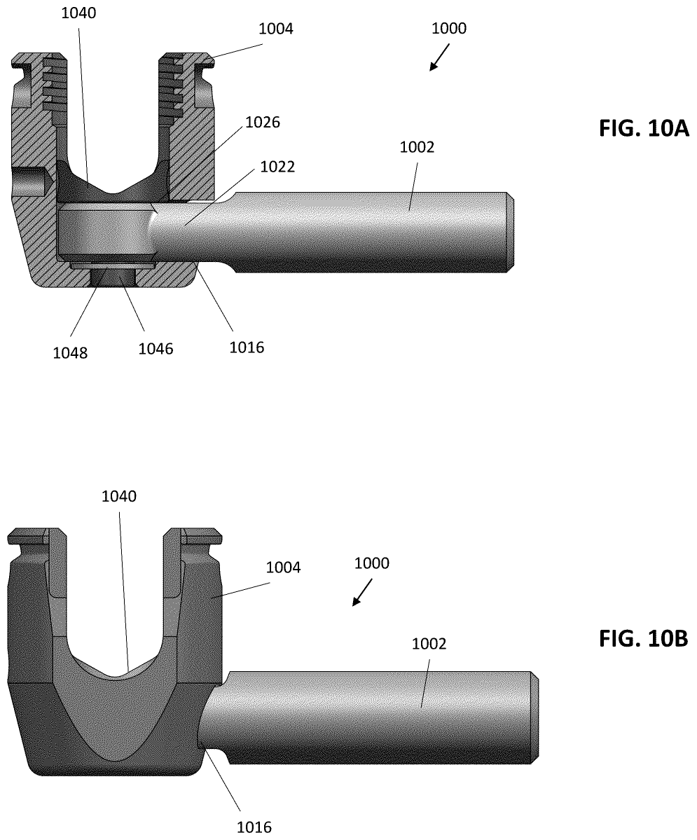

FIGS. 10A-10B illustrate one embodiment of a modular connector assembly 1000 in which the connector rod can pivot through a certain range of motion relative to the connector body. When implanted in a patient's spine, for example, this can result in an ability for the connector rod to pivot or move within the coronal plane. In these figures, side views of a modular connector assembly having a pivotable connector rod are shown. The modular connector assembly 1000 can include a connector body 1004, connector rod 1002, rod seat 1040, and spring or bias element 1048. The connector body 1004 can include any of the features described herein, including a socket 1016 for receiving the first end 1022 of the connector rod 1002. As described further below, the width of the socket 1016 can be greater than the width of the first end 1022 of the connector rod 1002 such that the connector rod 1002 can pivot between the bounding ends of the socket 1016. The connector rod 1002 can include at least one flat surface 1026 to facilitate coupling to the socket 1016 of the connector body 1004 in a correct orientation and to resist relative rotation of the connector rod relative to the connector body outside of the pivoting motion permitted by the wider socket. The spring 1048 can be positioned within the connector body 1004 such that a bore in the spring (not pictured) aligns with the bore of the connector rod 1002 and the end bore of the connector body 1004 when the connector rod 1002 is coupled to the connector body 1004. As such, a distal post 1046 of the rod seat 1044 can pass through the corresponding bore in the spring 1048, as well as the bore of the connector rod 1002, and end slot of the connector body 1004 when the rod seat 1040 is coupled to the modular connector system 1000. The spring 1048 can impart a pre-load or drag force onto the connector rod 1002 such that pivoting the connector rod 1002 relative to the connector body 1004 requires overcoming the pre-load or drag force.

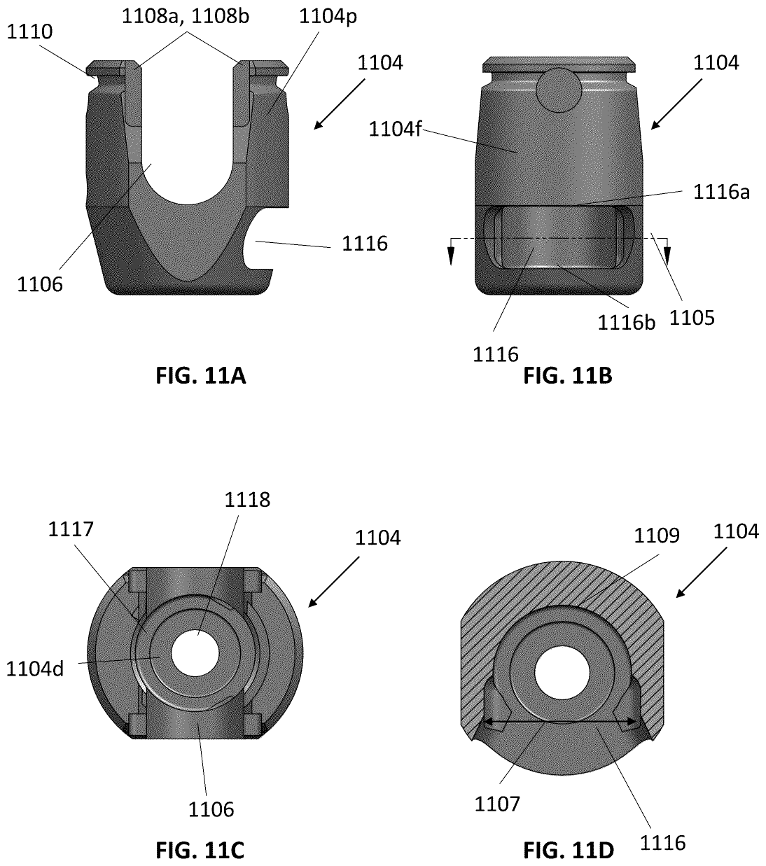

FIGS. 11A-11D illustrate one embodiment of a connector body 1104 that is similar to the connector body 1004 of FIGS. 10A-10B in greater detail. Similar to the embodiments of FIGS. 7A-7D, the connector body of FIG. 11A includes a pair of spaced apart arms 1108a, 1108b at the proximal end 1104p of the connector body 1104 that form a U-shaped recess 1106. The outer surface of each spaced apart arm 1108a, 1108b includes a top-notch feature 1110 for engaging various instruments. Near the distal end of the connector body is a socket 1116 for receiving a connector rod. In FIG. 11B, the front end 1104f of the connector body 1104 is shown, including the socket 1116. The socket 1116 formed in the connector body 1104 has a profile that enables the pivoting movement of the rod relative to the connector body 1104. For example, the socket 1116 can have an elongated oval shape that is greater in width than the width of a connector rod, allowing space within the socket 1116 for the connector rod to pivot. Comparing the width 1107 of the socket 1116 to the width 707 of the socket 716 in FIGS. 7A-7D also highlights this distinction between the embodiments. The socket 1116 can include at least one flat surface 1116a that corresponds to a flat surface on a first end of the connector rod. In some embodiments, the socket 1116 can include opposing proximal-distal flat surfaces 1116a, 1116b that correspond to opposing flat surfaces on the first end of the connector rod. A dotted line 1105 represents a cross section of the connector body shown in FIG. 11D.

FIG. 11C shows a top view of the connector body of FIG. 11A. The pair of spaced apart arms form a recess 1106 within the connector body 1104. The connector body 1104 can include an end slot or bore 1118 on the distal end 1104d of the connector body 1104. In addition, the distal inner surface of the connector body 1104 can include a recess 1117 surrounding the bore 1118 that can receive the spring 1048 or other element configured to impart a pre-load or drag force onto the connector rod. FIG. 11D shows the cross section view from the dotted line 1105 of FIG. 11B. As shown, the socket 1116 does not extend through both sides of the connector body 1104 and an inner surface of the connector body 1104 opposite the opening of the socket 1116 can have a curved sidewall 1109. Comparing the socket 1116 shown in FIGS. 11B and 11D to that shown in FIGS. 7B and 7D, for example, highlights the larger opening width 1107 of the connector body 1104 to allow for a pivoting range of motion between the connector rod 1102 and connector body 1104.

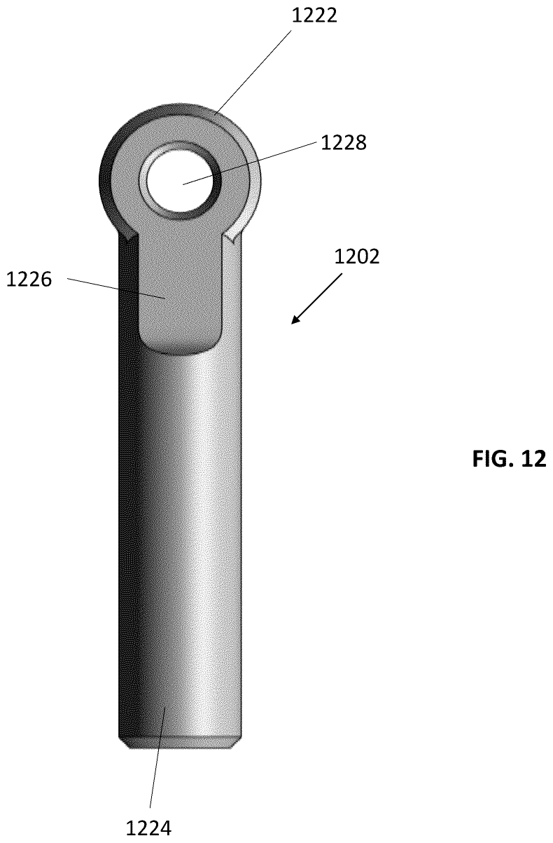

FIG. 12 illustrates one embodiment of a connector rod that is similar to the connector rod 1002 of FIGS. 10A-10B in greater detail. Its outer shape can be configured to match with the contour of the socket 1116 formed in the connector body 1104, as shown in FIGS. 11B and 11D, in order to allow pivoting movement between the connector rod and the connector body. For example, the connector rod 1202 can have a first end 1222 that has a curved shape to match the curved inner surface 1109 of the connector body 1104. The curved shape of the first end 1222 can have a diameter that is greater than a diameter of the remainder of the connector rod 1202, which can help secure the first end 1222 within the connector body 1104. The first end 1222 of the connector rod 1202 can have at least one flat surface 1226 to match the shape of the socket 1116 in the connector body 1104, which can help ensure proper assembly, provide a bearing surface to interface with a rod seat (e.g., rod seat 1040), and resist unwanted relative movement between the two components. The first end 1222 can also include a slot or bore 1228 for receiving a rod seat therethrough. A second end 1224 of the connector rod 1202 can be substantially cylindrical in shape, though a variety of other shapes and sizes are also possible.

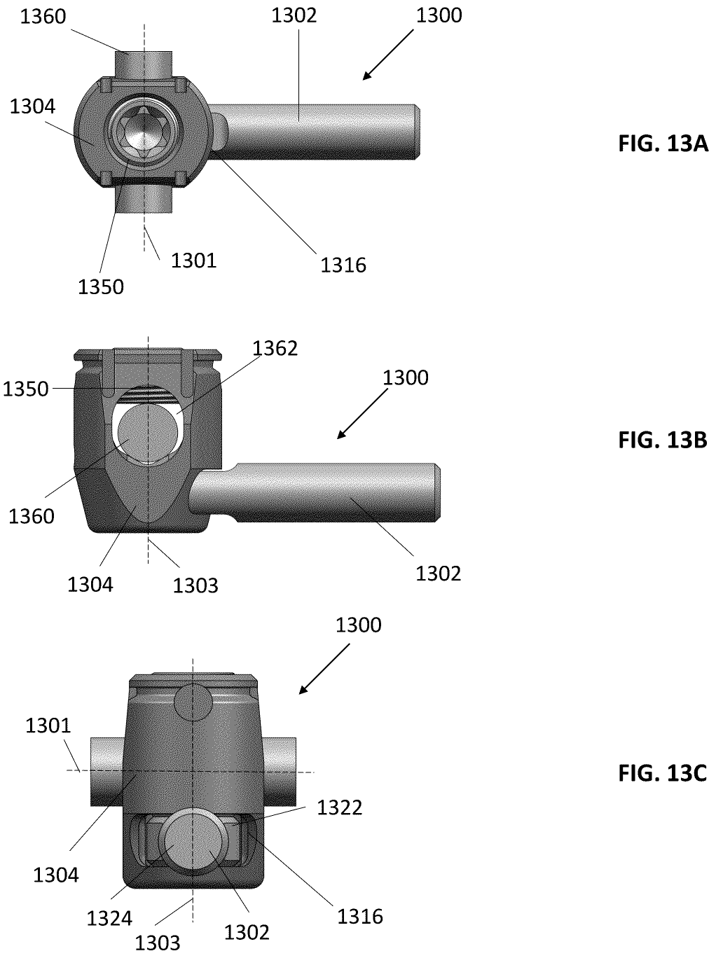

FIGS. 13A-13C illustrate one example of an alternative modular connector body type that can be utilized. In particular, the illustrated connector body 1304 is a closed-top body (a so-called “O” type connector) having a socket 1316 allowing for a pivoting connector rod 1302 to extend transverse to an axis 1301 of a rod capture bore 1362 formed in the body of the connector 1304. Such a connector body 1304 captures a rod 1360 by having it introduced along its longitudinal axis through the rod capture bore 1362, rather than top-loaded into a U-shaped rod seat, as is possible in the above-described “U” type embodiments. The rod 1360 is then secured in the rod capture bore 1362 with a set screw 1350. This embodiment can be similar to the connector shown in FIGS. 9A-9C, but can provide for the pivoting range of motion of the rod described with regard to the connector assemblies of FIGS. 10A-12. FIG. 13A shows a top view of the modular connector system 1300 having a set screw 1350 secure a rod 1360 in the connector body 1304. FIG. 13B shows a side view of the modular connector assembly 1300, illustrating the rod 1360 within the rod capture bore 1362 and the rod 1302 extending perpendicular to the central longitudinal axis 1303 of the connector body 1304. FIG. 13C depicts the socket 1316 in the connector body 1304 that is wide enough to permit pivoting motion of the connector rod about the longitudinal axis 1303 of the connector body, as well as the spherical shape of a first end 1322 of the connector rod seated within the connector body that is wider than a diameter of a second end 1324 of the rod that extends therefrom. FIG. 13C also shows the perpendicular configurations of the rod capture bore axis 1301, connector body longitudinal axis 1303, and longitudinal axis of the connector rod (extending out of the plane of the page in this figure). The connector rod 1302 can depart from this perpendicular configuration at least with respect to the axis 1301, however, in that it can pivot about the axis 1303.

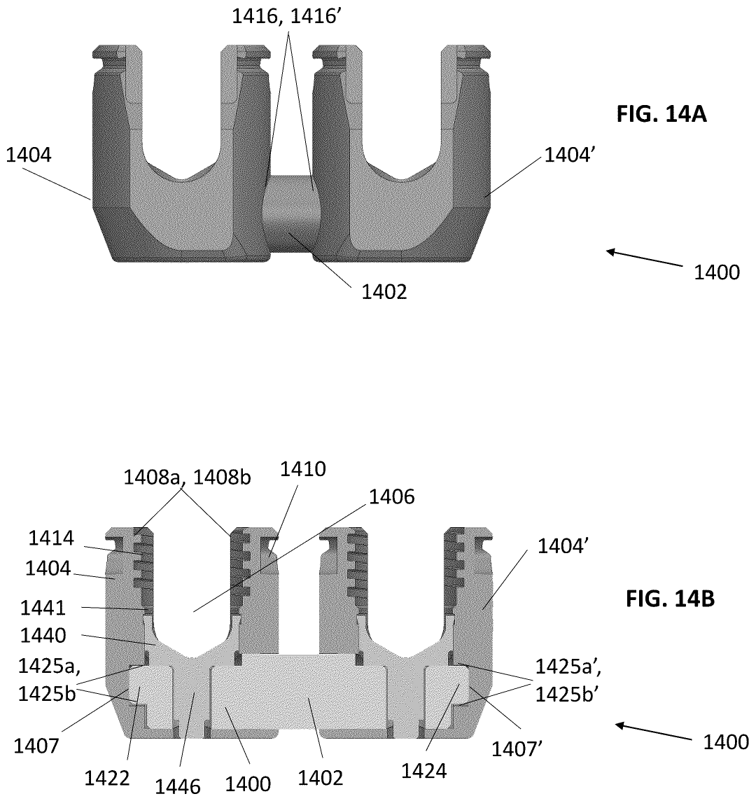

The above described modular assemblies have generally been described with regard to “O-R” or “U-R” type connectors, where a connector rod makes up one half of an assembly and a “U” or “O” type connector body makes up the other half of the assembly. Other connector assembly configurations are possible, however, and the FIGS. 14A-24B disclosure certain alternatives. The present disclosure contemplates a modular platform for assembling connectors having any of a variety of configurations. The disclosed embodiments are examples that could be combined in various manners, and with other types of connector components, within the scope of this disclosure.

FIGS. 14A-14C illustrate one embodiment of a modular connector assembly having first and second connector bodies coupled by a rod. The connector bodies can be similar to the “U” type connector bodies described above. Accordingly, the connector assembly shown in FIGS. 14A-14C can be referred to as a “U-U” connector. FIG. 14A is a side view of a modular connector assembly 1400 having a first connector body 1404 and an opposing second connector body 1404′. A statically-positioned connector rod 1402 can be coupled to each of the first and second connector bodies 1404, 1404′ via opposed ends of the rod 1402 extending into respective sockets 1416, 1416′ on each connector body 1404, 1404′ and being captured within the connector bodies by respective rod seats 1440, 1440′. Each of the first and second connector bodies 1404, 1404′ can include any of the features described herein.

For example, in FIG. 14B, a cross sectional view of the modular connector assembly 1400 of FIG. 14A shows a second connector body 1404′ having substantially the same features as a first connector body 1404. The first connector body 1404 can include a proximal pair of spaced apart arms 1408a, 1408b forming a U-shaped opening or recess 1406 in the first connector body 1404. The external surface of the pair of spaced apart arms 1408a, 1408b can include a top-notch feature 1410 for coupling to various instruments. The internal surface of the pair of spaced apart arms 1408a, 1408b can include threads 1414 for coupling to a set screw. A retention surface or lip 1441 can be positioned distal to the threads 1414 and can prevent proximal movement of the rod seat 1440 once the rod seat 1440 is inserted distally into the first connector body 1404 such that a proximal end of the rod seat passes distally beyond the lip 1441. The rod seat 1440 can have a distal post 1446 that extends distally through a bore formed in the connector rod 1402 and the connector body 1404. The rod seat 1440 can include any of the features described herein. For example, the ends of the rod 1402 can include opposed flat surfaces 1425a, 1425b, 1425a′, 1425b′ at each end thereof that can be received within complementary-shaped recesses in the connector bodies 1404, 1404′ to assist with proper assembly and prevent unwanted relative movement between the components. Further, the first and second connector bodies 1404, 1404′ can have internal recesses 1407, 1407′ configured to accept the terminal ends of the first and second ends 1422, 1424 of the connector rod 1402. The terminal ends 1422, 1424 can include one or more flat surfaces that can interface with flat surfaces formed in the recesses 1407, 1407′, respectively, to help resist relative movement or bending of the connector rod 1402 and the connector bodies 1404, 1404′, such as rotation of the connector rod 1402 relative to the connector bodies 1404, 1404′ about a longitudinal axis of the rod. Generally speaking, the configuration of the features of the second connector body 1404′ can substantially mirror the features of the first connector body 1404. For example, the second connector body 1404′ can couple to the second end 1424 of the connector rod 1402 when the features of the second end 1424 mirror the features of the first end 1422 of the connector rod 1402 relative to the first and second connector bodies, respectively.

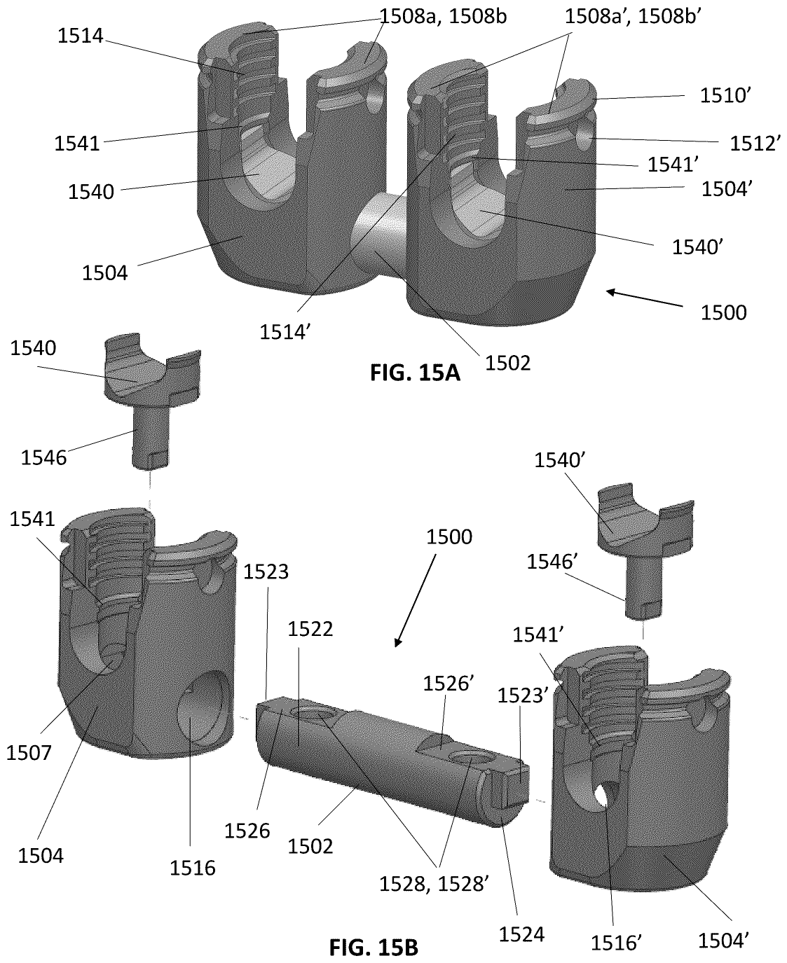

FIG. 15A shows a perspective view of an embodiment of a modular connector assembly 1500 having first and second connector bodies 1504, 1504′. The embodiment of FIG. 15A can be similar or the same as the embodiment shown in FIGS. 14A and 14B. A first connector body 1504 can include a first proximal pair of spaced apart arms 1508a, 1508b creating a U-shaped opening 1506 therebetween. The outer surface of the first pair of spaced apart arms 1508a, 1508b can include a top-notch feature 1510 and a proximal rocker feature 1512. The internal surface of the first pair of spaced apart arms 1508a, 1508b can include threads 1514 and a retention feature 1541. The retention feature 1541 can lock a first rod seat 1540 within the first connector body 1504 to prevent proximal removal thereof after the rod seat has been inserted distally beyond the retention feature. A connector rod 1502 connects the first connector body 1504 and the second connector body 1504′. The connector rod 1502 is secured to the connector bodies 1504, 1504′ with rod seats 1540, 1540′. The second connector body 1504′ includes the same features as the first connector body 1504, i.e., a second pair of spaced apart arms 1508a′, 1508b′ having a top-notch feature 1510′, a proximal rocker feature 1512′, threads 1514′, and a retention feature 1541′.

FIG. 15B shows an exploded view of the modular connector assembly 1500 of FIG. 15A. In FIG. 15B, further details of the components of the modular connector assembly can be seen. For example, the first connector body 1504 includes a socket 1516 for receiving a corresponding first end 1522 of the connector rod 1502. The first connector body 1504 also includes an internal recess 1507 having a shape that correlates to the terminus 1523 of the first end 1522 of the connector rod 1502, such that the terminus 1523 couples to the internal recess 1507 when the connector rod 1502 is inserted into the first connector body 1504. The terminus 1523 can include one or more flat surfaces that can interface with flat surfaces formed in the recess 1507 to help resist relative movement or bending of the connector rod 1502 and the connector body 1504, such as rotation of the connector rod 1502 relative to the connector body 1504 about a longitudinal axis of the rod. The first end 1522 of the connector rod 1502 further includes a flat surface 1526 extending toward a midline of the rod to aid coupling to the first connector body 1504 and resisting certain movement, such as rotation of the rod 1502 relative to the connector body 1504 about a longitudinal axis of the rod. A bore 1528 in the first end 1522 of the connector rod 1502 allows the distal post 1546 of a first rod seat 1540 to pass through the first end 1522 of the connector rod 1502.

The second connector body 1504′ includes substantially the same features as the first connector body 1504. A second end 1524 of the connector rod 1502 includes a flat surface 1526′ to aid coupling to the second connector body 1504′ when inserted through the socket 1516′ and resisting certain movement, such as rotation of the rod 1502 relative to the connector body 1504 about a longitudinal axis of the rod. A bore 1528′ in the second end 1524 of the connector rod 1502 allows the distal post 1546′ of a second rod seat 1540′ to pass through the second end 1524 of the connector rod 1502 and the second connector body 1504′. The terminus 1523′ of the second end 1524 of the connector rod 1502 correlates to an internal recess (not shown in FIG. 15, see recess 1407 in FIG. 14B) in the second connector body 1504′ and aids coupling of the second end 1524 of the connector rod 1502 when the connector rod 1502 is inserted into the second connector body 1504′. For example, the terminus 1523′ can include one or more flat surfaces that can interface with flat surfaces formed in the recess of the connector body 1504′ to help resist relative movement or bending of the connector rod 1502 and the connector body 1504′, such as rotation of the connector rod 1502 relative to the connector body 1504′ about a longitudinal axis of the rod.

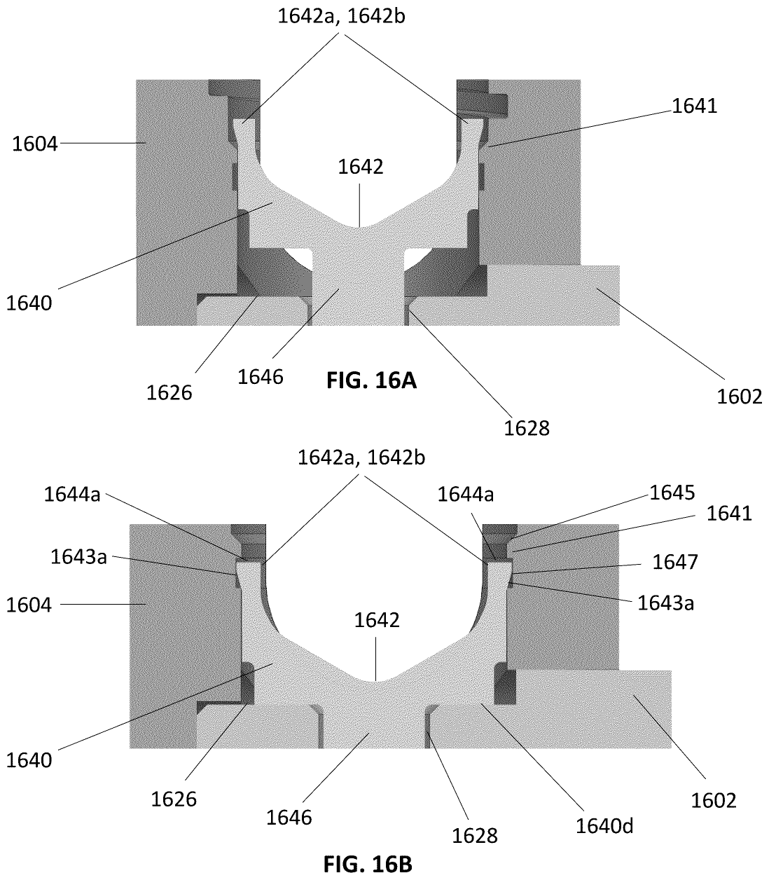

In assembly of the modular connector system 1500, the first end 1522 of the connector rod 1502 can be inserted laterally into the socket 1516 of the first connector body 1504. After insertion of the first end 1522 of the connector rod 1502, the first rod seat 1540 can be advanced distally into the U-shaped opening 1506 of the first connector body 1504. The distal post 1546 on the first rod seat 1540 can pass through the bore 1528 in the first end 1522 of the connector rod 1502 to prevent separation of the connector rod 1502 from the first connector body 1504 (see cross-sectional view of FIG. 14B). The rod seat 1540 can have a transition fit through the bore 1528 in the connector rod 1502. The first rod seat 1540 can be locked into place within the first connector body 1504 by snapping into the retention feature 1541, as explained in more detail below in connection with FIGS. 16A and 16B. The second end 1524 of the connector rod 1502 can be inserted into the socket 1516′ of the second connector body 1504′. The second rod seat 1540′ can be advanced distally into the U-shaped opening 1506′ of the second connector body 1504′, the distal post 1546′ of the second rod seat 1540′ passing through the bore 1528′ in the second end 1524 of the connector rod 1502. The rod seat 1540′ can have a transition fit through the bore 1528′ in the connector rod 1502. The second rod seat 1540′ can be locked into place within the second connector body 1504′ in the same manner as the first rod seat 1540, i.e., by snapping into the retention feature 1541′, as explained below in connection with FIGS. 16A and 16B. In some embodiments, assembly can begin with insertion of the components relating to the second connector body 1504′. In some embodiments, the first and second ends 1522, 1524 of the connector rod 1502 can be inserted into the first and second connector bodies 1504, 1504′ before the first and second rod seats 1540, 1540′ are advanced distally into the respective connector bodies 1504, 1504′.

FIGS. 16A-16B illustrate a detailed view of one embodiment of a rod seat and connector body like those shown in FIG. 15A-15B. In FIG. 16A, the rod seat 1640 is partially inserted into the connector body such that its proximal end is disposed proximal to the retention feature 1641 of the connector body. In FIG. 16B, the rod seat is down fully inserted into the connector body such that its proximal end is disposed distal to the retention feature 1641 of the connector body. The rod seat 1640 includes a proximal end having a substantially U- or V-shaped rod-seating surface 1642 that can seat a spinal rod. In some embodiments, the shape of the proximal rod-seating surface can exactly match a diameter of a rod to be used with the rod seat. In other embodiments, a substantially V-shaped surface, or a surface having opposed planar surfaces angled toward one another (that may or may not be meet at a central point) can accommodate the use of various rods having different diameters. The proximal ends of the rod seat can form resilient arms 1642a, 1642b that can include outward projections 1643a, 1643b with distal-facing ramps and planar proximal-facing surfaces 1644a, 1644b. In use, as the rod seat is advanced distally from the position of FIG. 16A to the position of FIG. 16B, the outward projections 1643a, 1643b with distal-facing ramps can come into contact with a proximal-facing ramped surface 1645 of the retention feature 1641. As the rod seat is advanced further distally, the resilient arms 1642a, 1642b can deflect inward until they pass distally beyond the retention feature 1641, which can be a perpendicular lip or shoulder formed just distal to the proximal-facing ramped surface 1645. Once past the retention feature 1641, the resilient arms 1642a, 1642b can return outward to their resting position and the outward projections 1643a, 1643b can be disposed within a groove 1647 in the inner sidewall of the connector body. At this point, the rod seat can be locked against withdrawal proximally from the connector body, as the planar proximal-facing surfaces 1644a, 1644b of the rod seat would interfere with the distal-facing surface of the retention feature 1641 if the rod seat is sufficiently moved proximally. A proximally-distally extending height of the groove 1647 can allow for some movement of the rod seat relative to the connector body while preventing complete removal of the rod seat. The rod seat 1640 includes a distal post 1646 configured to pass through a bore 1628 in the connector rod 1602. In FIG. 16B, the rod seat 1640 is coupled to the connector body 1604 and the connector rod 1602. Particularly, the planar proximal ends 1642a, 1642b are in contact with the retention feature 1641 of the connector body 1604, thereby preventing proximal movement of the rod seat 1640 within the connector body 1604. A distal surface 1640d of the rod seat 1640 is in contact with a flat surface 1626 of the connector rod 1602. The distal post 1646 of the rod seat 1640 is securely passed through the bore 1628 of the connector rod 1602.

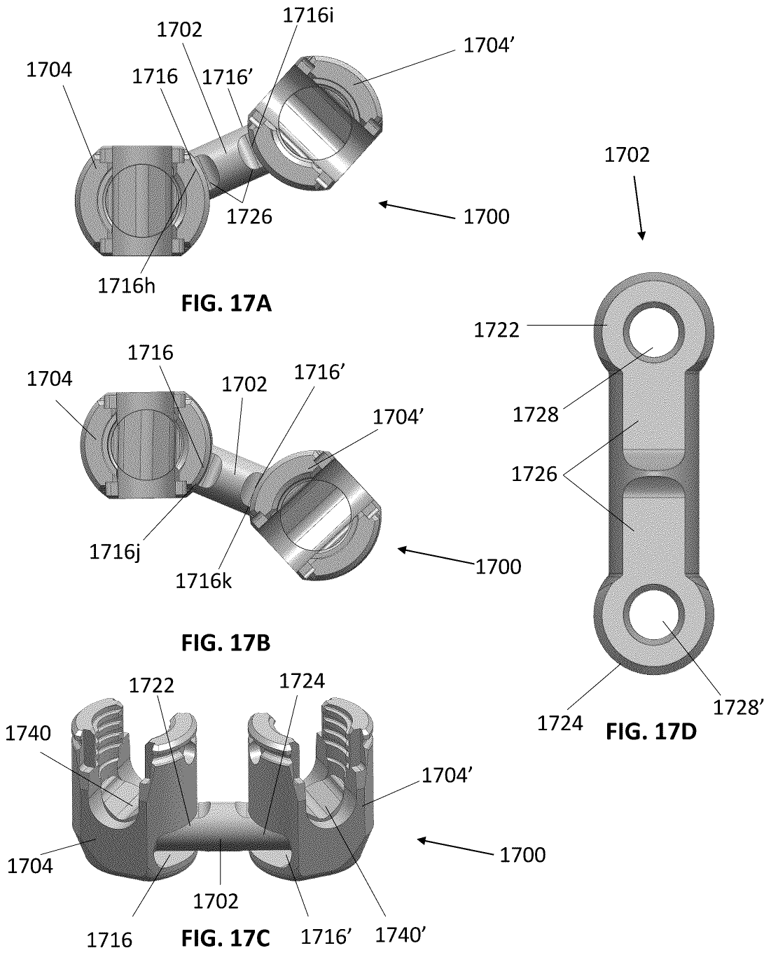

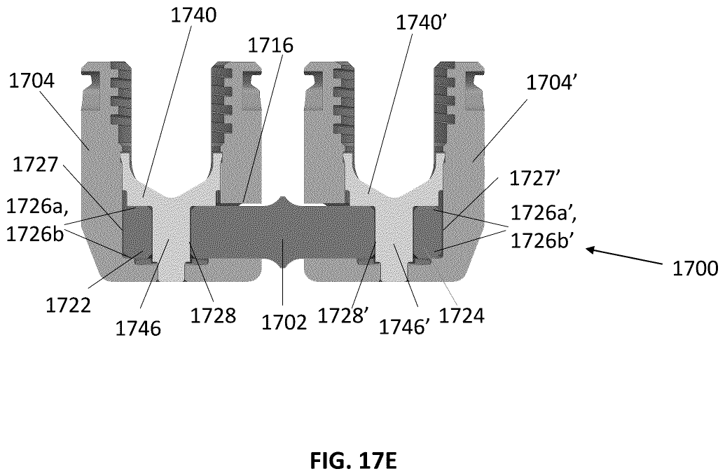

FIGS. 17A-17B illustrate an embodiment of the modular connector assembly of the present disclosure having a first and second connector bodies and a pivotable connector rod. FIG. 17A shows a top view of a modular assembly 1700 having a first connector body 1704 and a second connector body 1704′. Each of the first and second connector bodies 1704, 1704′ can include any of the features described herein, particularly a socket 1716, 1716′ for receiving a first or second end of a connector rod 1702, respectively. The width of each socket 1716, 1716′ can be greater than the width of a mid-portion of the connector rod 1702, such that the connector rod 1702 can pivot through the width of each socket 1716, 1716′. Each of the first and second ends of the connector rod 1702 can include at least one flat surface 1726 for complementary coupling to the socket 1716, 1716′ of each of the first and second connector bodies 1704, 1704′. A spring (not shown in FIG. 17, see spring 1048 in FIG. 10A) within each of the connector bodies 1704, 1704′ can impart a pre-load or drag force on the connector rod 1702 such that moving the connector rod 1702 relative to the first or second connector body 1704, 1704′ requires overcoming the pre-load or drag force. As shown in FIG. 17A, each of the first and second ends of the connector rod 1702 can be independently pivotable, i.e., the first end can pivot within the socket 1716 of the first connector body 1704 simultaneously to, and differently from, the second end within the socket 1716′ of the second connector body 1704′. In some embodiments, however, only one of the connectors can be capable of pivoting motion while the other is fixed. FIG. 17B further depicts a top view of the pivotable connections of the modular connector assembly 1700 of FIG. 17A. Comparing FIG. 17A to FIG. 17B, the second end of the connector rod 1702 is capable of pivoting from a first side 1716i of the socket 1716′ of the second connector body 1704′ in FIG. 17A to a second side 1716k of the socket 1716′ in FIG. 17B. Further, the first end of the connector rod 1702 is capable of pivoting from a first side 1716h of the socket 1716 of the first connector body 1704 in FIG. 17A to a second side 1716j of the socket 1716 of the first connector body 1704 in FIG. 17B.

FIG. 17C shows a perspective view of the modular connector system 1700 of FIG. 17A in further detail. In FIG. 17C, a first rod seat 1740 couples the first end 1722 of the connector rod 1702 to the first connector body 1704. A second rod seat 1740′ couples the second end 1724 of the connector rod 1702 to the second connector body 1704′. The sockets 1716, 1716′ of the first and second connector bodies 1704, 1704′ each have a width greater than the width of a central portion of the connector rod 1702, thus allowing the connector rod 1702 to pivot in a limited range within each of the sockets 1716, 1716′ of the first and second connector bodies 1704, 1704′.

FIG. 17D shows a top view of the connector rod 1702 of the modular connector system 1700 of FIG. 17A. Each of the first and second ends 1722, 1724 of the connector rod 1702 has a shape substantially similar to the first end of the connector rod of FIG. 12. Particularly, each of the first end 1722 and the second end 1724 of the connector rod 1702 has an outer shape configured to match with the contour of each socket formed in a connector body. The first end 1722 and the second end 1724 can be have a curved shape with a diameter that is greater than a diameter of a central portion of the connector rod 1702. Each of the first end 1722 and the second end 1724 can have at least one flat surface 1726 to match the shape of the sockets of the first and second connector bodies. Each of the first and second ends 1722, 1724 can also include a bore 1728, 1728′ for receiving a distal post of a rod seat.

FIG. 17E shows a cross sectional view of the modular assembly 1700. Illustrated in the figure is the configuration of the connector rod 1702 with a first end 1722 coupled to the first connector body 1704 and a second end 1724 coupled to the second connector body 1704′. The first end 1722 and the second end 1724 of the connector rod 1702 can have substantially mirrored features, as noted above. For example, the first end 1722 can include a pair of opposed flat surfaces 1726a, 1726b that can match the shape of an inner surface of the first connector body 1704. The first end 1722 can also have an outer shape, such as a curved surface 1727 extending between the flat surfaces 1726a, 1726b, configured to match a contour of a socket formed in the connector body 1704. This curved surface can have a diameter that is greater than a diameter of a central portion of the connector rod 1702. The first end 1722 can include a bore 1728 that can allow a distal post 1746 of a rod seat 1740 to pass therethrough and couple the connector rod 1702 and the first connector body 1704. Similarly, the second end 1724 of the connector rod 1702 can include a pair of opposed flat surfaces 1726a′, 1726b′ to aid coupling to the second connector body 1704′ and resisting certain movement, such as rotation of the rod 1702 relative to the connector body 1704′ about a longitudinal axis of the rod. The second end 1724 can also have an outer shape, such as a curved surface 1727′ extending between the flat surfaces 1726a′, 1726b′, configured to match a contour of a socket formed in the connector body 1704′. This curved surface can have a diameter that is greater than a diameter of a central portion of the connector rod 1702. The second end 1724 can also include a bore 1728′ for passing a distal post 1746′ of the rod seat 1740′ through the connector rod 1702 and coupling the rod seat 1740′ to the second connector body 1704′.

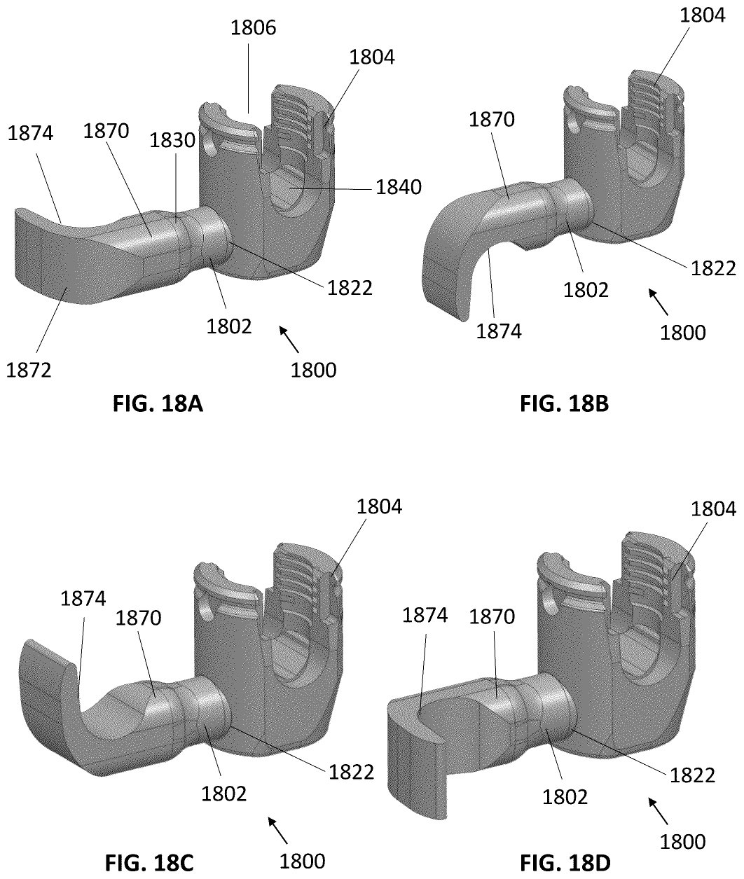

FIGS. 18A-18D illustrate another embodiment of a modular connector assembly. In the illustrated embodiment of FIG. 18A, the assembly 1800 includes a first connector body 1804 having a substantially U-shaped opening 1806, and a second connector body 1870 that is substantially hook shaped. The first connector body 1804 can be similar to those described, for example, in FIGS. 14A-16B, and can include any of the features of the present disclosure. The second connector body 1870, or hook body, includes a convex side 1872 opposing a concave side 1874 that creates the hook shape. The concave side 1874 can be configured to couple to a rod, bar, or piece of patient anatomy. The hook body 1870 can include a connector rod portion 1802 on a second end of the connector rod 1802 such that the hook body 1870 and the second end of the connector rod 1802 are unitary or integrally formed, though in some embodiments they can be formed of separate components coupled to one another. A taper 1830 can connect the hook body 1870 to the connector rod portion 1802 such that the hook body 1870 can have different dimensions than a diameter of the connector rod portion 1802.

The connector rod portion 1802 can include a first end 1822 configured to couple to the first connector body 1804. The first end 1822 of the connector rod portion 1802 can include a bore (not shown, see bore 228 in FIG. 2) which the distal post of a rod seat 1840 can pass through and thus couple the hook body 1870 to the first connector body 1804. In some embodiments, the bore in the first end 1822 of the connector rod portion 1802 can run parallel to the plane of the hook. In some embodiments, the bore can be perpendicular to the plane of the hook. In certain embodiments, the first end 1822 of the connector rod portion 1802 can include both a parallel bore and a perpendicular bore to the plane of the hook, such that the parallel bore and the perpendicular bore intersect at a right angle within the first end 1822 of the connector rod portion 1802. In FIG. 18A, the first end 1822 of the connector rod 1802 includes a bore that is perpendicular to the plane of the hook, which allows the concave side 1874 of the hook body 1870 to face a medial or lateral direction with respect to the first connector body 1804.

FIGS. 18B-18D show the hook body of FIG. 18A in alternative orientations when connected to the first connector body. In FIG. 18B, the first end 1822 of the connector rod portion 1802 includes a bore that is parallel to the plane of the hook, which allows the concave side 1874 of the hook body 1870 to face a distal direction with respect to the first connector body 1804. In FIG. 18C, the hook body 1870 and connector rod portion 1802 of FIG. 18B are rotated 180 degrees such that the concave side 1874 of the hook body 1870 faces a proximal direction with respect to the first connector body 1804. In FIG. 18D, the hook body 1870 and connector rod portion 1802 of FIG. 18A are rotated 180 degrees such that the concave side 1874 of the hook body 1870 faces a medial or lateral direction opposite that of FIG. 18A. The modular assembly 1800 of FIGS. 18A-18D can also be referred to as a “U-H” connector.

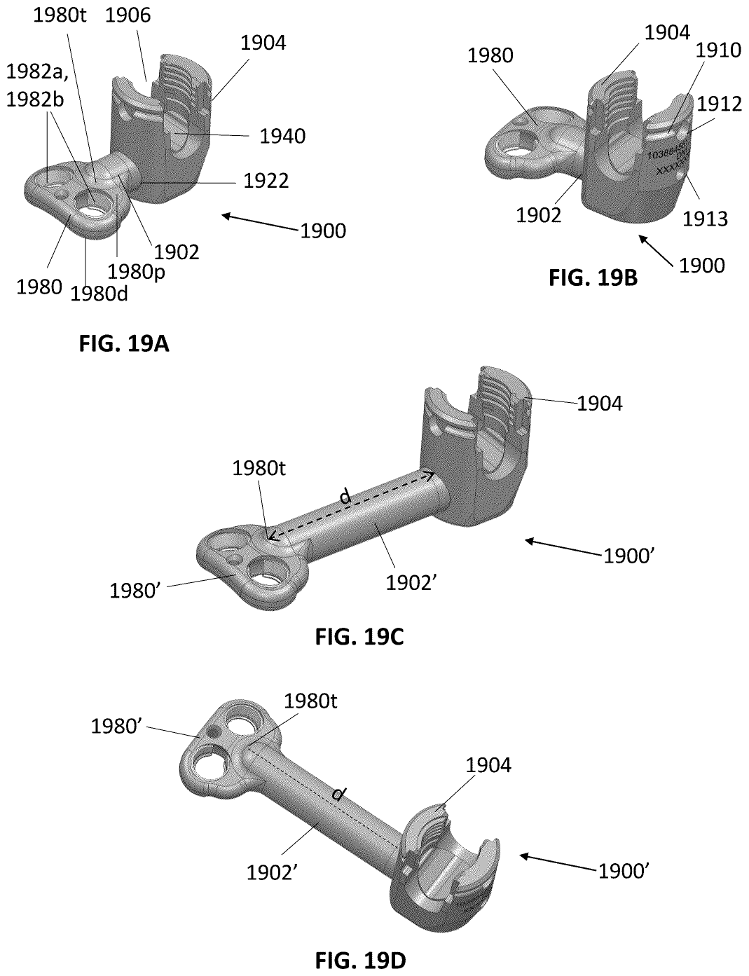

FIGS. 19A-19D illustrate another embodiment of a modular connector assembly having additional bores for the insertion of auxiliary fixation or multipoint bone screws. In the illustrated embodiment of FIG. 19A, the assembly 1900 includes a first connector body 1904 having a substantially U-shaped opening 1906, and a second connector body 1980 that is a multipoint or auxiliary fixation adapter. The first connector body 1904 is similar to those described, for example, in FIGS. 14A-16B, and can include any of the features of the present disclosure. The second connector body 1980, or multipoint adapter, can have a flattened, substantially oval shape, with proximal and distal sides 1980p, 1980d. The multipoint adapter 1980 can include at least one anchor bore 1982a capable of receiving a bone anchor. In some embodiments, the multipoint adapter 1980 can include a pair of anchor bores 1982a, 1982b that pass through in a proximal-distal direction. The pair of anchor bores 1982a, 1982b can be parallel, such that insertion of a pair of bone anchors into the pair of anchor bores 1982a, 1982b would allow the pair of anchors to be parallel when coupled to a bone. In other embodiments, the anchor bores 1982a, 1982b can be angled in the same manner or in different manners such that auxiliary fixation screws inserted therethrough can pass into a patient's anatomy with different trajectories. Still further, in some embodiments the anchor bores 1982a, 1982b can include threads that can accept screws at different trajectories. Additional details on possible multipoint or auxiliary fixation bore features and screws for use with same can be found in U.S. Pat. Nos. 9,962,192; 10,898,232; 11,426,210; and 11,304,728. The entire contents of each of these patents are incorporated herein by reference.

The multipoint adapter 1980 can include a connector rod portion 1902, as a unitary or integrally formed portion or a separate component coupled therewith. The proximal and distal surfaces 1980p, 1980d of the multipoint adapter 1980 can transition into a cylindrical shape of the connector rod portion 1902 at a transition point 1980t. The connector rod portion 1902 can include a first end 1922 configured to couple to the first connector body 1904. The first end 1922 can include one or more flat portions and a bore (not shown, as described above) that can allow a distal post of a rod seat 1940 to pass through the connector rod 1902 and couple it to the first connector body 1904.

FIG. 19B shows an alternative view of the modular connector assembly of FIG. 19A. In this view, it can be seen that the first connector body 1904 can include features discussed throughout the disclosure, such as a top-notch feature 1910, a proximal rocker feature 1912, and a distal rocker feature 1913.

FIGS. 19C and 19D show an alternative embodiment of the modular connector assembly of FIG. 19A that includes an elongated connector rod 1902′. Comparing the modular connector assembly 1900 of FIG. 19A to the assembly 1900′ of FIG. 19C, the connector rod 1902′ has a greater length that can allow for coupling of the multipoint adapter 1980′ to different anatomy than the multipoint adapter 1980 of FIG. 19A. In FIGS. 19C and 19D, the first connector body 1904 and the multipoint adapter 1980′ include substantially the same features as the first connector body 1904 and multipoint adapter 1980 in FIGS. 19A-19B. One distinction is that the transition point 1980t of FIGS. 19C and 19D is at a distance d from the first connector body 1904 that is longer than the distance separating the transition point 1980t from the connector body 1904 in FIGS. 19A and 19B. The modular systems 1900, 1900′ of FIGS. 19A-19D can also be referred to as a “U-A” connector.

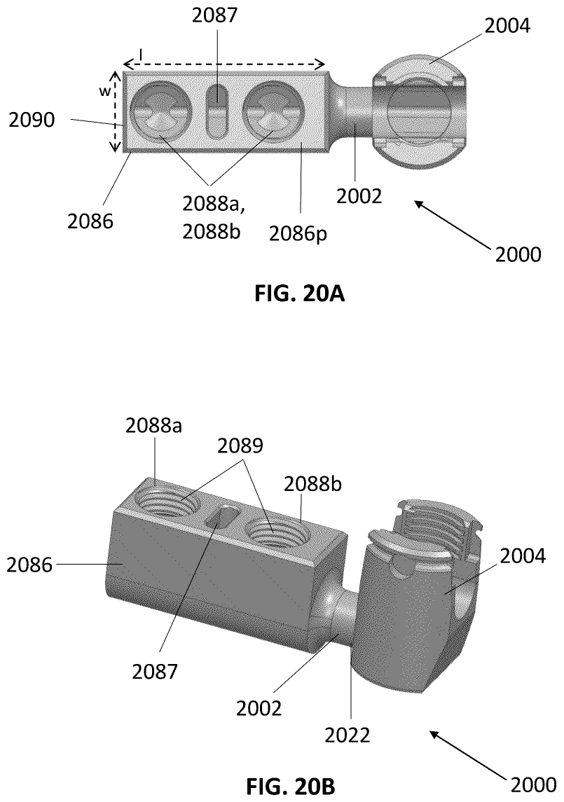

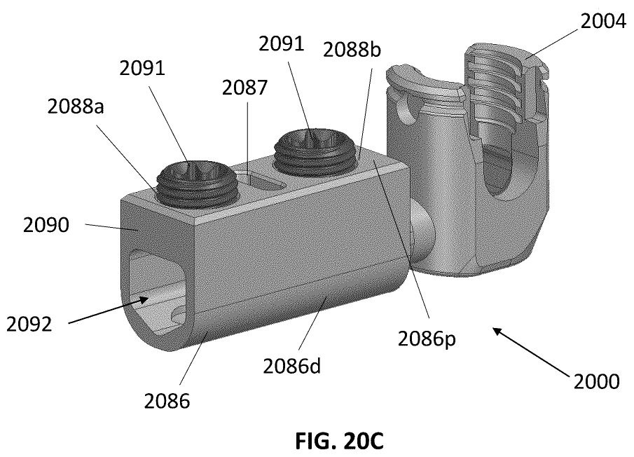

FIGS. 20A and 20B illustrate another embodiment of a modular connector assembly of the present disclosure. In FIG. 20A, a top view of a modular connector assembly 2000 includes a first connector body 2004 that is similar to those described, for example, in FIGS. 14A-16B, and can include any of the features of the present disclosure. The first connector body 2004 can be coupled to a second connector body 2086 that is an in-line connector via a connector rod portion 2002 of the in-line connector 2086. The in-line connector 2086 can be used, for example, in revision surgery to extend a previous spinal fixation construct in a cranial or caudal direction by coupling to a terminal end of a spinal fixation rod. In particular, a surface 2090 opposite the connector rod portion 2002 can include an opening 2092 (see FIG. 20C) formed therein that can receive a rod therethrough. The in-line connector 2086 can have a substantially rectangular shape with a substantially planar proximal surface 2086p and a curved distal surface 2086d. The in-line connector 2086 can have a length l that is greater than its width w. The proximal surface 2086p of the in-line connector 2086 can include a pair of bores 2088a, 2088b. The bores 2088a, 2088b can have threads 2089 and be configured to receive set screws 2091 (see FIG. 20C) used to fix any rod inserted into the in-line connector against movement relative to the connector 2086. In some embodiments, the proximal surface 2086p of the in-line connector 2086 can also include an window 2087 to view a rod inserted into the in-line connector 2086. The in-line connector 2086 can be integrally formed with, or coupled to, the connector rod portion 2002.

In FIGS. 20B and 20C, perspective views of the modular assembly of FIG. 20A are shown. Each of the pair of bores 2088a, 2088b can include threads 2089 for coupling to a screw or other threaded component, such as the set screw 2091 mentioned above. The connector rod portion 2002 can include a first end 2022 configured to couple to the first connector body 2004. The first end 2022 can include a bore (not shown, as described above) that can allow a distal post of a rod seat to pass through the connector rod 2002 and couple the rod to the first connector body 2004. The modular assembly 2000 of FIGS. 20A-20C can also be referred to as a “U-I” connector.

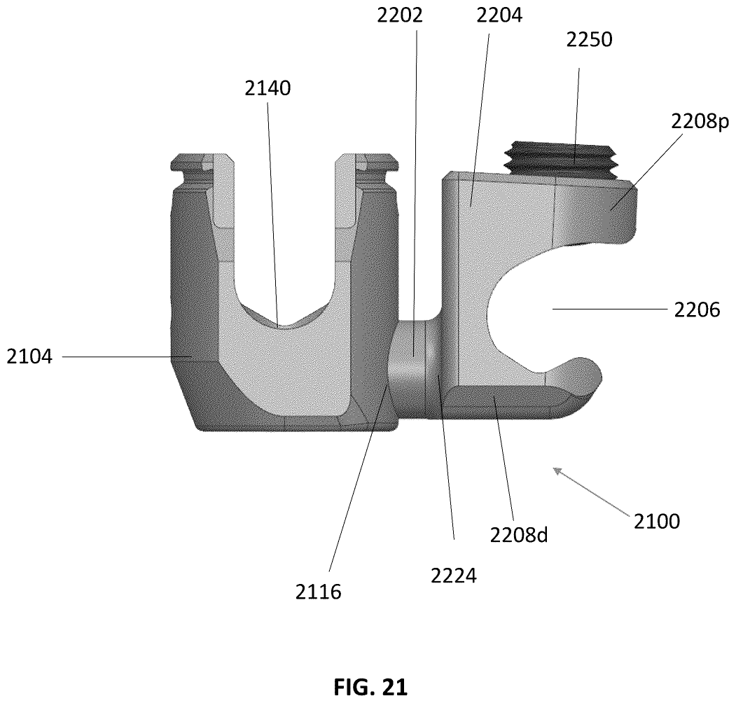

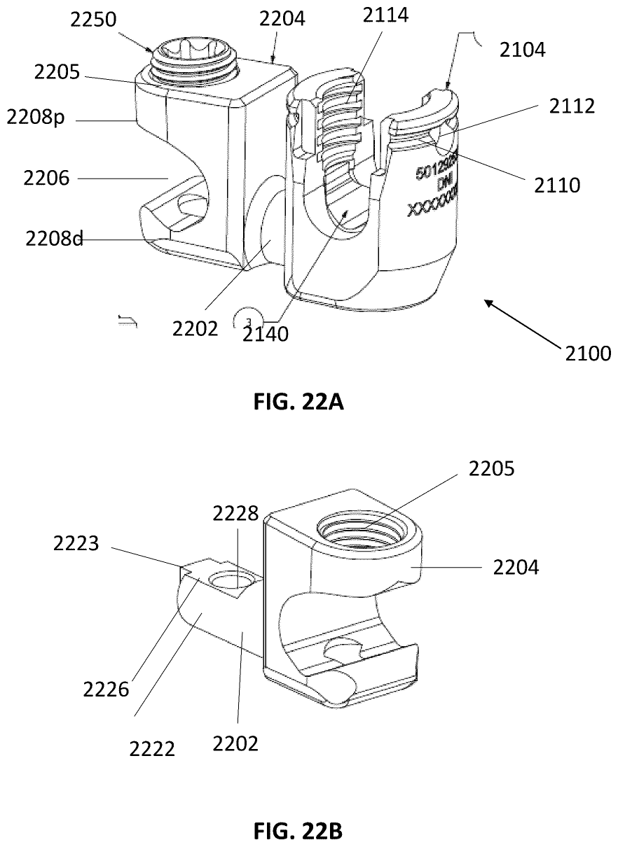

FIG. 21 illustrates an embodiment of a modular connector assembly having a first connector body and an alternatively shaped second connector body. The modular connector assembly 2100 includes a first connector body 2104 capable of having all of the features of the present disclosure. The second connector body 2204 can include a pair of spaced apart arms 2208p, 2208d, the first arm 2208p extending from a proximal side of the second connector body 2204 and the second arm 2208d extending from a distal side of the second connector body 2204. The pair of spaced apart arms 2208p, 2208d can define a substantially C-shaped opening 2206 in the second connector body 2204. The C-shaped opening 2206 can be capable of receiving a spinal fixation element or rod within the second connector body 2204. The first arm 2208p can include a bore (not shown in FIG. 22A, see bore 2205 in FIG. 22B) capable of receiving a set screw 2250. The set screw 2250 can be threaded into the first arm 2208p such that it can secures a rod within the C-shaped opening 2206.

The C-shaped opening 2206 can allow a rod to be coupled to the second connector body 2204 at an angle that is perpendicular to a connector rod portion 2202. The connector rod portion 2202 can connects the second connector body 2204 to the first connector body 2104 via a socket 2116 in the first connector body 2104. In some embodiments, the second connector body 2204 can be integrally formed with, or coupled to, the connector rod portion 2202 on a second end 2224 of the connector rod 2202 opposite the C-shaped opening 2206. The connector rod portion 2202 can include a first end 2222 (not shown in FIG. 21, as described above and see FIG. 22B) configured to couple to the first connector body 2104. The first end 2222 can include a bore (not shown in FIG. 21, as described above and see FIG. 22B) that allows a distal post of a rod seat 2140 to pass through the connector rod 2202 and couple the second connector 2204 to the first connector body 2104.

FIGS. 22A and 22B show the various components of the modular connector assembly of FIG. 21. As shown in FIG. 22A, the first connector body 2104 can include features discussed in the present disclosure, including a top-notch feature 2110, a proximal rocker feature 2112, and internal threading 2114. A rod seat 2140 can couple the connector rod portion 2202 to the first connector body 2104 by passing a distal post (not shown, as described above) of the rod seat 2140 through the bore 2228 (see FIG. 22B) in the first end of the connector rod 2202. The second connector body 2204 can have a pair of spaced apart arms 2208p, 2208d defining the substantially C-shaped opening 2206. A set screw 2250 can be threaded into the bore 2205 in the first arm 2208p on the second connector body 2204.

FIG. 22B shows that the second connector body 2204 and the connector rod portion 2202 can be an integrated unit. The bore 2205 in the first arm of the second connector body can be threaded for coupling to the set screw 2250. The connector rod portion 2202 can have a first end 2222 that can include at least one flat surface 2226 for aiding coupling and securing of the second connector body 2204 to the first connector body 2104. A bore 2228 in the first end 2222 of the connector rod portion 2202 can allow the distal post of the rod seat to pass through the connector rod 2202. A terminus 2223 on the first end 2222 of the connector rod 2202 can have a shape complementary to an internal recess (see, e.g., recess 1407 in FIG. 14B) in the first connector body to aid coupling of the connector rod portion 2202 within the first connector body. The modular assembly of FIGS. 21-22B can also be referred to as a “U-C” connector.

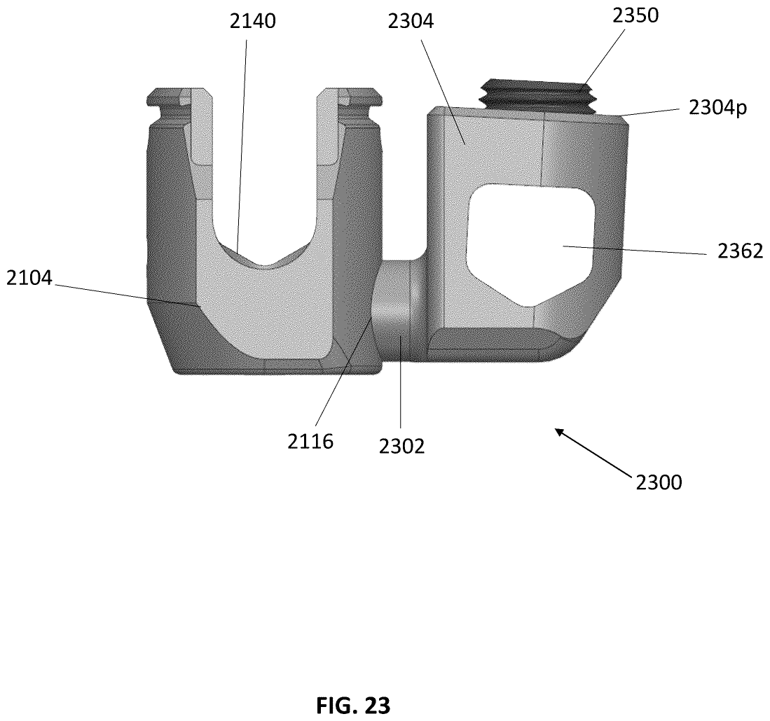

FIG. 23 illustrates an embodiment of a modular connector assembly having a first connector body and an alternatively shaped second connector body. The modular connector system 2300 includes a first connector body 2104 capable of having any of the features of the present disclosure. The second connector body 2304 can include a closed-top body similar to the closed-top body of FIGS. 9A-9C. The closed-top connector body 2304 can include a rod capture bore 2362 formed along a horizontal axis of the closed-top connector body 2304. A bore in the proximal end 2304p of the closed-top body 2304 can allow for coupling to a set screw 2350. The closed-top connector body 2304 can capture a spinal rod by sliding the spinal rod along its longitudinal axis through the rod capture bore 2362 and securing the spinal rod with the set screw 2350, rather than top-load into a U-shaped rod seat, as is possible with the first connector body 2104. The closed-top connector body 2304 can be integrally formed with, or coupled to, a connector rod 2302. The connector rod 2302 can connect the closed-top connector body 2304 to the first connector body 2104. A first end of the connector rod 2302 can be inserted into a socket 2116 of the first connector body 2104 and secured within the first connector body 2104 by passing a distal post of a rod seat 2140 through a bore 2328 in the first end of the connector rod 2302.

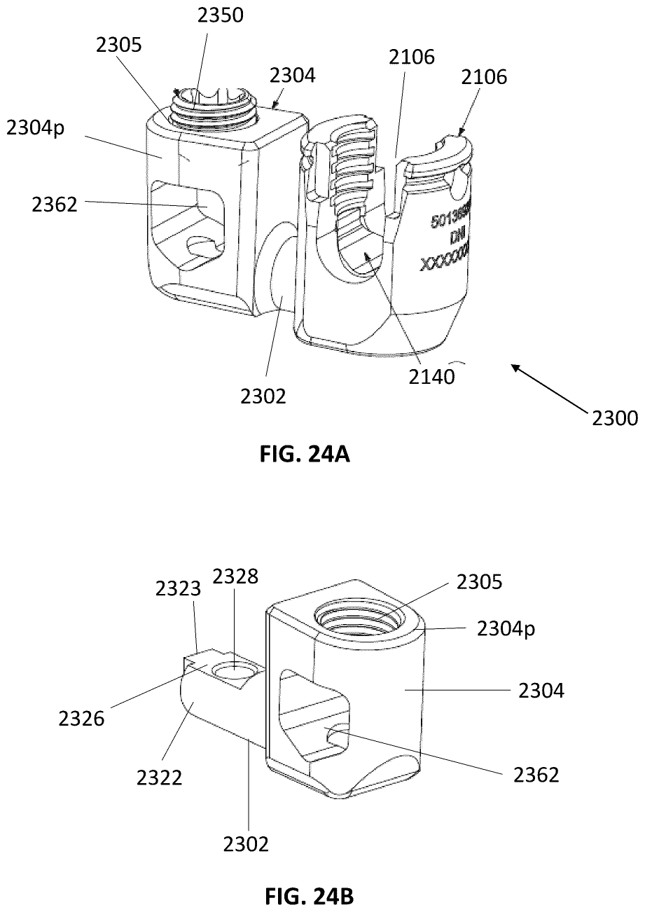

FIGS. 24A and 24B show the various components of the modular connector assembly of FIG. 23. In FIG. 24A, the second or closed-top connector body 2304 can be coupled to the first connector body 2104 via the connector rod portion 2302. The rod seat 2140 within the U-shaped opening 2106 of the first connector body 2104 can secure the first end of the connector rod 2302 within the first connector body 2104. The closed-top connector body 2304 can include a rod capture bore 2362 extending along an axis perpendicular to the longitudinal axis of the connector rod portion 2302. In FIG. 24B, the closed-top connector body 2304 is integrally formed with the connector rod portion 2302. The bore 2305 on the proximal end 2304p of the closed-top body 2304 can be threaded for coupling to the set screw 2350. The first end 2322 of the connector rod portion 2302 can include a flat surface 2326 to aid assembly and secure coupling to the first connector body. The bore 2328 in the first end 2322 of the connector rod 2302 can allow the distal post of the rod seat 2140 to pass through the connector rod 2302 and couple the second connector 2304 to the first connector body. A terminus 2323 on the first end 2322 of the connector rod portion 2302 can have a shape that is contoured to match an internal recess of the first connector body when the first end 2322 of the connector rod 2302 is inserted into the socket of the first connector body. The modular assembly of FIGS. 23-24B can also be referred to as a “U-O” connector.

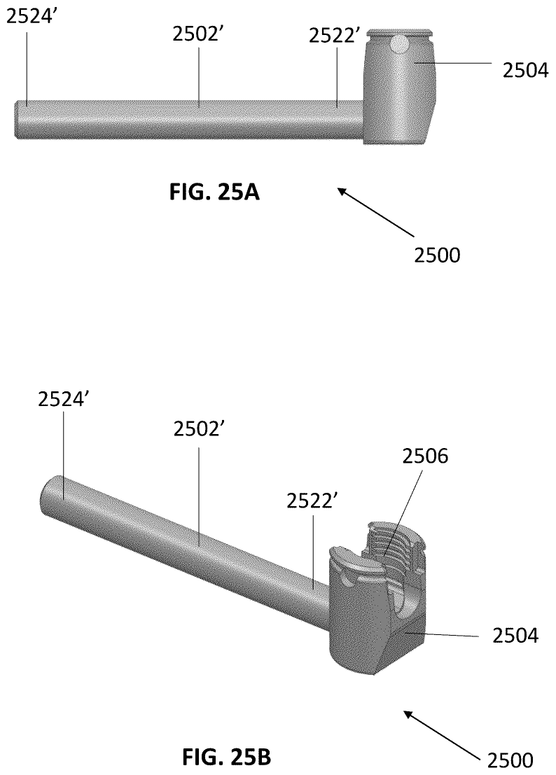

FIGS. 25A and 25B illustrate another embodiment of a modular connector assembly in the U-R configuration. Similarly to FIGS. 1A and 1B, the modular connector assembly 2500 includes a statically positioned connector rod 2502 extending from one side of a connector body 2504. The connector body 2504 can be configured to capture a spinal rod within a U-shaped recess 2506. The connector body 2504 can include all of the distinct engagement or attachment features described in the disclosure. In FIG. 25A, a side view of the modular connector system 2500 illustrates a longer connector rod 2502′ being utilized than in the modular connector system of FIG. 1A. Any of a variety of rod lengths can be utilized, including, for example, rod lengths between about 20 mm and about 200 mm. Different connector rod diameters can be utilized as well, including, for example, connector rod diameters of about 5.5 mm and about 6 mm, among others. FIG. 25B shows a perspective view of the modular connector system of FIG. 25A. As can be seen in FIG. 25B, the elongated connector rod 2502′ can omit a taper such that a first end 2522′ and a second end 2524′ of the connector rod 2502′ have a uniform diameter. Omission of the taper can help facilitate reception of the connector rod 2502′ into the socket formed in the connector body 2504. The components of the modular connector system of FIGS. 25A and 25B can include any of the features of the present disclosure.

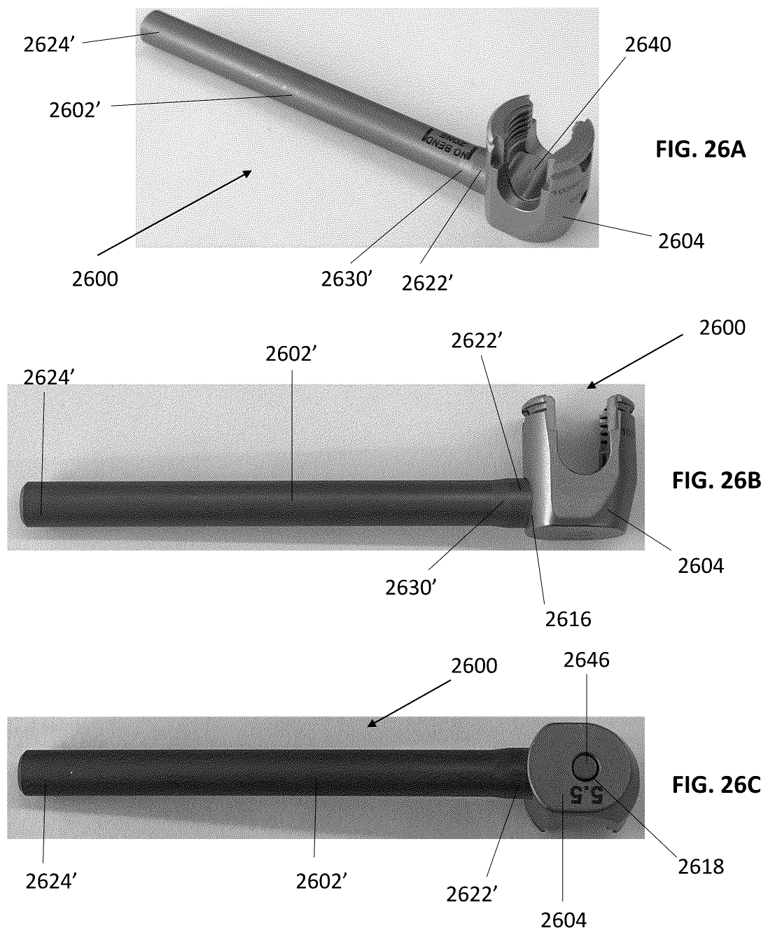

FIGS. 26A-26C illustrate another embodiment of a modular connector assembly in the U-R configuration. In FIG. 26A, the modular connector assembly 2600 can be substantially similar to the modular connector assembly of FIG. 25A, having an elongated connector rod 2602′. In FIG. 26A, however, the connector rod 2602′ can include a taper 2630′ between a first end 2622′ and a second end 2624′ of the connector rod 2602′. FIG. 26B shows a side view of the modular connector system of FIG. 26A having an elongated connector rod 2602′ with a taper 2630′. The first end 2622′ of the elongated connector rod 2602′ can be coupled to the connector body 2604 via a socket 2616 in the connector body 2604. A rod seat (see 2640 in FIG. 26A) can secure the first end 2622′ of the connector rod 2602′ within the connector body 2604. FIG. 26C shows a bottom view of the modular connector assembly of FIG. 26A. Particularly, FIG. 26C illustrates an end slot 2618 in a distal end 2604d of the connector body 2604. The distal post 2646 of the rod seat 2640 is situated within the end slot 2618, thereby securing the first end 2622′ of the connector rod 2602′ to the connector body 2604.

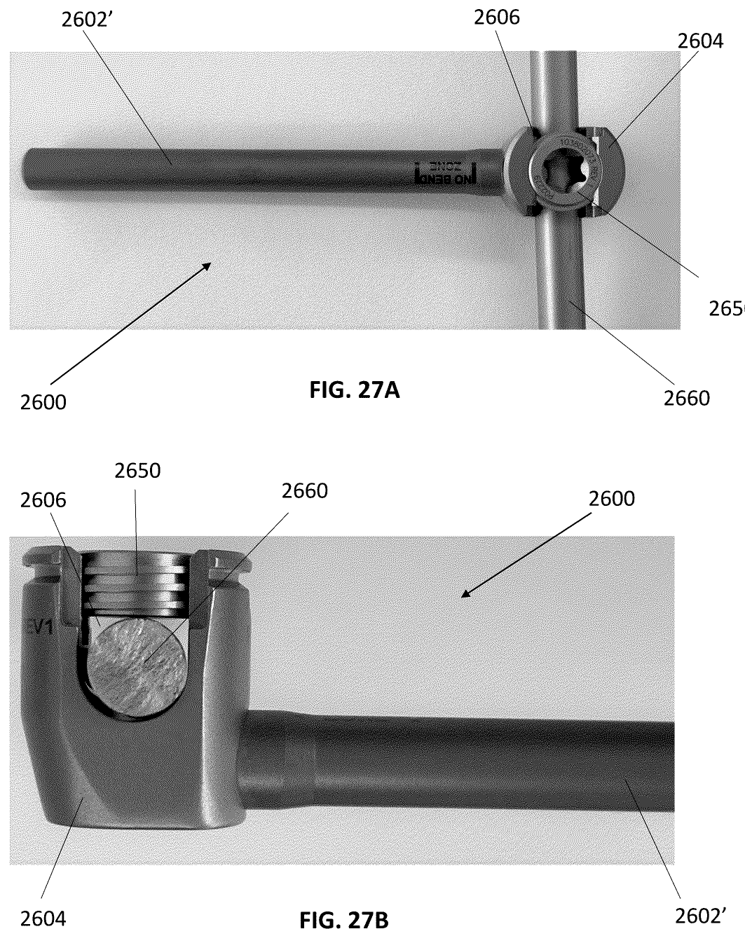

FIGS. 27A and 27B illustrate the modular connector assembly of FIG. 26A having a spinal rod coupled thereto. In FIG. 27A, a top view of the modular connector assembly 2600 shows the connector body 2604 coupled to the connector rod 2602′. A spinal rod 2660 is coupled to the connector body 2604 in the U-shaped opening 2606. The spinal rod 2660 is secured within the U-shaped opening 2606 by a set screw 2650. The spinal rod 2660 has a longitudinal axis that is substantially perpendicular to a longitudinal axis of the connector rod 2602′.

FIG. 27B shows a side view of the modular connector assembly of FIG. 26A having a spinal rod coupled thereto. The spinal rod 2660 is secured in the U-shaped opening 2606 of the connector body 2604 by a set screw 2650 having external threads that are complementary to the threads within the U-shaped opening 2606 of the connector body 2604 (i.e., on the internal surface of a pair of spaced apart arms). The spinal rod 2660 extends from the connector body 2604 at an axis perpendicular to the longitudinal axis of the connector rod 2602′.

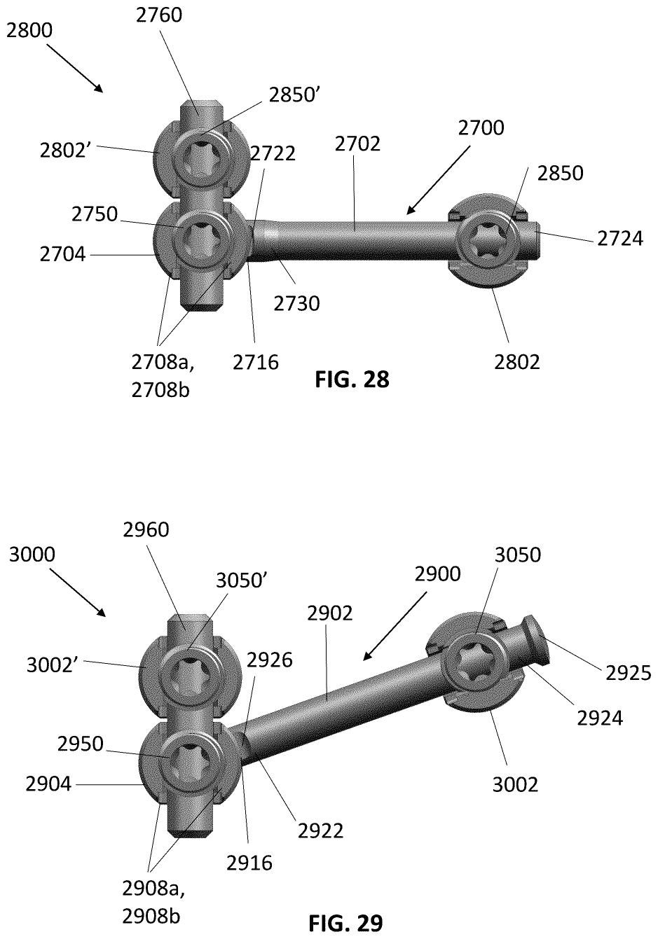

FIG. 28 illustrates one embodiment of a posterior fixation construct 2800 that can be created utilizing the above-described modular connector assemblies, though any of a variety of different constructs are also possible by combining the above-described modular connectors and other spinal fixation elements. In FIG. 28, a modular connector assembly 2700 in a U-R configuration is utilized. A first bone anchor 2802 can be coupled to a second end 2724 of a connector rod 2702 by securing the second end 2724 of the connector rod 2702 in a recess of the first bone anchor 2802 with a set screw 2850. A first end 2722 of the connector rod 2702 is coupled to a connector body 2704 via a socket 2716 in the connector body 2704. The connector rod 2702 includes a taper 2730 between the first end 2722 and the second end 2724. A spinal rod 2760 is secured within the U-shaped opening formed by a pair of spaced apart arms 2708a, 2708b on the proximal end of the connector body 2704. The spinal rod 2760 is secured with a set screw 2750. A second bone anchor 2802′ can be coupled to the spinal rod 2760 abutting the connector body 2704. The spinal rod 2760 is secured within a recess of the second bone anchor 2802′ by a set screw 2850′. The orientation of the spinal rod 2760 is perpendicular to a longitudinal axis of the connector rod 2702. Any of a variety of different constructs are possible by combining the above-described modular connectors and other spinal fixation elements in different combinations, including combinations of connector bodies with statically positioned connector rods, pivoting connector rods, flanged connector rods, connector rods of different diameters, connector rods of different lengths, different styles of connector bodies (e.g., top-loading, closed/side loading, etc.), etc.

FIG. 29 illustrates a posterior fixation construct 3000 similar to the construct of FIG. 28. Particularly in FIG. 29, a second end 2924 of a connector rod includes a flanged terminus 2925. A first bone anchor 3002 is coupled to the second end 2924 of the connector rod 2902 via a set screw 3050. A first end 2922 of the connector rod 2902 extends into a socket 2916 in a connector body 2904 of the modular connector assembly 2900. The socket 2916 of the connector body 2904 has an enlarged width to allow the connector rod 2902 to pivot while coupled to the connector body 2904. As shown in FIG. 29, the connector rod 2902 is pivoted to a first side of the socket 2916, though pivoting in an opposite direction is also possible. A spinal rod 2960 is secured within a U-shaped opening formed by a pair of spaced apart arms 2908a, 2908b on the proximal end of the connector body 2904. The spinal rod 2960 is secured with a set screw 2950. A second bone anchor 3002′ can be coupled to the spinal rod 2960 abutting the connector body 2904. The second bone anchor 3002′ is secured to the spinal rod 2960 by a set screw 3050′. Unlike the spinal rod in FIG. 28, the spinal rod 2960 of FIG. 29 extends at an oblique angle to the connector rod 2902 due to the connector rod 2902 being pivoted to a first side of the socket 2916.

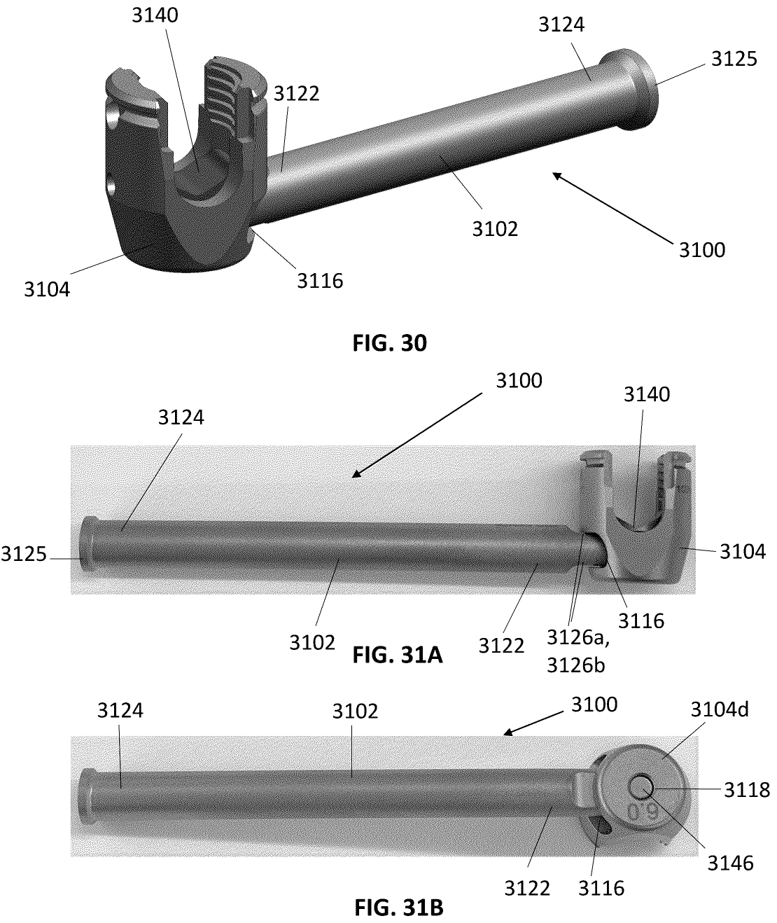

FIG. 30 illustrates a perspective view of a U-R modular connector assembly of the present disclosure. The modular connector assembly 3100 includes a connector body 3104, and elongated connector rod 3102, and a rod seat 3140. The connector body 3104 can include any of the features described herein, such as with the connector body of FIG. 1A. The connector rod 3102 can include a first end 3122 configured to couple to the connector body 3104 and a second end 3124 having a flanged terminus 3125. A socket 3116 in the connector body 3104 can have an enlarged width that allows the connector rod 3102 to pivot within the socket 3116 of the connector body 3104 when the connector rod 3102 is coupled to the connector body 3104 via the rod seat 3140. A distal post of the rod seat 3140 can couple the first end 3122 of the connector rod 3102 to the connector body 3104 by extending through a bore in the first end 3122 of the connector rod 3102. The rod seat 3140 can be locked within the connector body 3104 by a press fit connection or swage connection, as described above.

FIGS. 31A and 31B illustrate additional views of the U-R modular connector assembly of FIG. 30. In FIG. 31A, a side view of the modular connector assembly 3100 shows the elongated connector rod 3102 having a first end 3122 and a flange 3125 on the second end 3124. The first end 3122 includes a pair of opposed flat surfaces 3126a, 3126b that match the contour of the socket 3116 in the connector body 3104. The pair of opposed flat surfaces 3126a, 3126b can also facilitate pivoting of the connector rod 3102 within the socket 3116. The connector rod 3102 is secured within the connector body 3104 via the distal post of the rod seat 3140. FIG. 31B shows a bottom view of the modular connector system of FIG. 30. In this view, the extended width of the socket 3116 of the connector body 3104 relative to that of the connector rod 3102 is visible. The width of the socket 3116 can allow the connector rod 3102 a limited range of motion to pivot while coupled to the connector body 3104. A distal end 3104d of the connector body includes a bore 3118. The bore 3118 accepts the distal post 3146 of the rod seat when the rod seat is coupled to the connector rod 3102 and the connector body 3104.

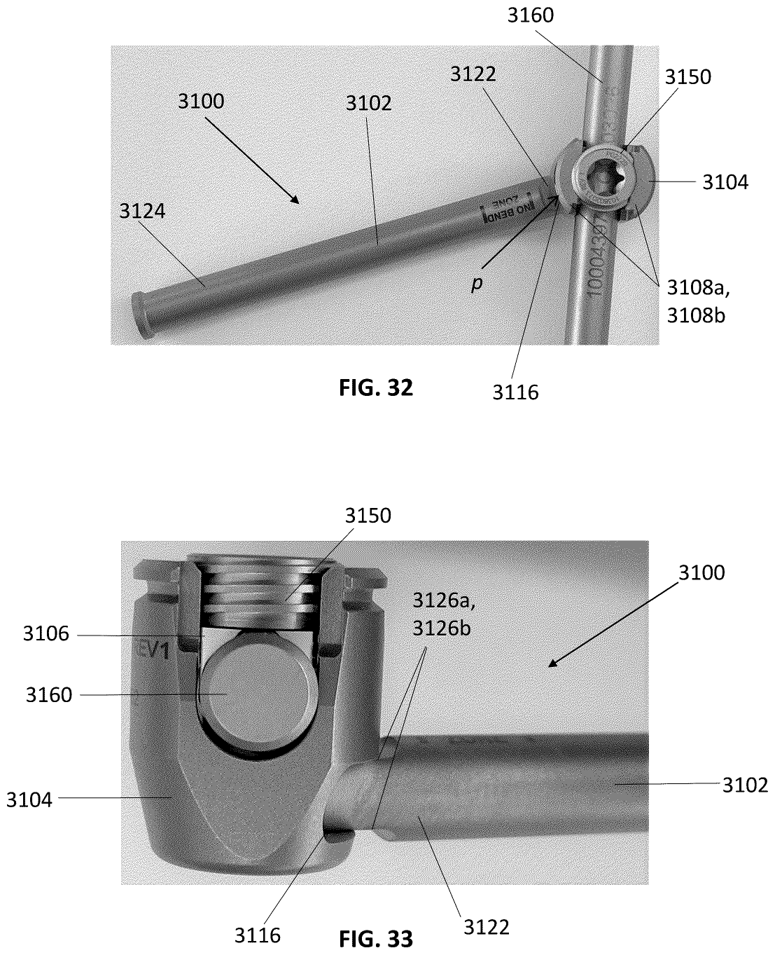

FIG. 32 illustrates a top view of the modular connector assembly of FIG. 30 coupled to a spinal rod. As noted above, the modular connector system 3100 includes the connector body 3104, the rod seat (not shown in FIG. 32), the connector rod 3102, and a set screw 3150. A spinal rod 3160 is positioned within the U-shaped opening defined by the pair of spaced apart arms 3108a, 3108b of the connector body 3104. The spinal rod 3160 is secured to the connector body 3104 with the set screw 3150. The connector rod 3102 is coupled to the connector body 3104 through the socket 3116 in the connector body 3104 and secured to the connector body 3104 by the distal post of the rod seat passing through the bore in the first end 3122 of the connector rod 3102. As can be seen in FIG. 32, the connector rod 3102 is pivoted until one side of the rod contacts an edge of the socket 3116 at point p. In this pivoted configuration, the connector rod 3102 has a longitudinal axis that extends obliquely to a longitudinal axis of the spinal rod 3160. In some embodiments, the connector assembly can be configured to provide up to about 48 degrees of pivoting motion between the connector rod 3102 and the connector body 3104, i.e., about 24 degrees in either direction relative to a configuration in which a longitudinal axis of the connector rod 3102 is perpendicular to a longitudinal axis of the spinal rod 3160. In other embodiments, the socket 3116 of the connector body 3104 and/or rod 3102 can be differently configured to provide more or less range of pivoting motion therebetween.

FIG. 33 illustrates a side view of the assembly of FIG. 30 coupled to a spinal rod. From this view, the spinal rod 3160 is positioned within the U-shaped opening 3106 of the connector body 3104 and secured to the connector body 3104 by the set screw 3150. The set screw 3150 includes external threads to facilitate coupling to the connector body 3104. The first end 3122 of the connector rod is shown having opposing flat surfaces 3126a, 3126b to facilitate coupling to the socket 3116 and internal surfaces of the connector body 3104. The socket 3116 has an enlarged width to allow the connector rod 3102 to pivot as shown in FIG. 32.

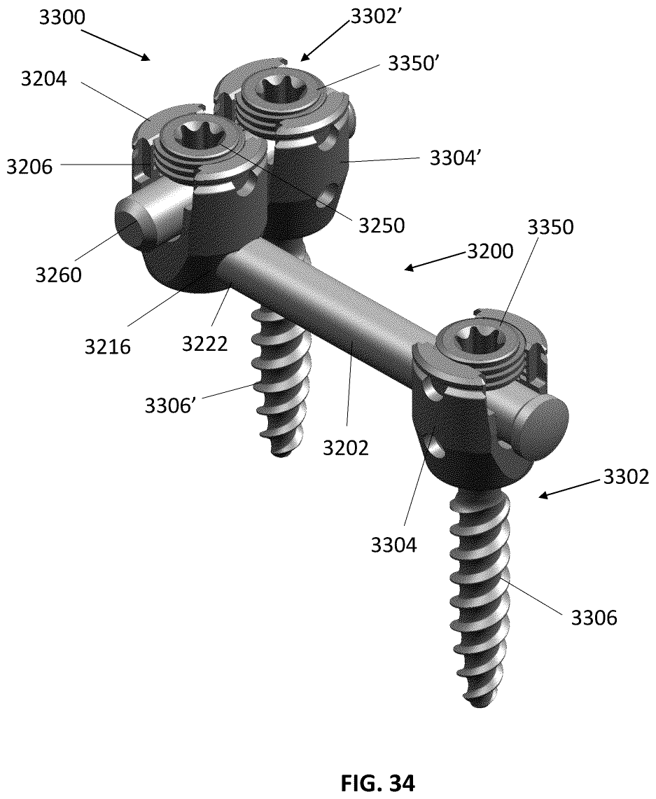

FIG. 34 illustrates a perspective view of one embodiment of a fixation construct 3300 that can be created using a modular connector assembly 3200. In FIG. 34, a U-R modular connector assembly 3200 includes a connector body 3204, a rod seat (not shown, see rod seat 1640 in FIG. 16A), a set screw 3250, and a connector rod 3202. The connector rod 3202 can include any of the features described in the disclosure, such as with the connector rod of FIG. 29 (i.e., a first end, a flanged second end, a bore, etc.). The second end 3224 of the connector rod 3202 is coupled to a first bone anchor 3302 via a set screw 3350. The first bone anchor 3302 includes a receiving head 3304 for accepting the second end 3224 of the connector rod 3202, and a threaded distal shank 3306 for coupling the bone anchor 3302 to bone. A spinal rod 3260 is coupled to the connector body 3204, the spinal rod 3260 positioned within the U-shaped opening 3206 of the connector body 3204 and secured to the connector body 3204 with a set screw 3250. The connector body 3204 can include any of the features disclosed herein, including but not limited to, threads, a top-notch feature, etc. The connector body 3204 includes a socket 3216 having an extended width such that the connector rod 3202 can pivot about the width of the socket 3216. Also coupled to the spinal rod 3260 is a second bone anchor 3302′. The second bone anchor 3302′ includes a receiver head 3304′ and a threaded distal post 3306′, similar to those of the first bone anchor 3302. The second bone anchor 3302′ is coupled to the spinal rod 3260 by a set screw 3350′. The second bone anchor 3302′ is positioned adjacent to the connector body 3204 of the modular connector system 3200, however, the second bone anchor 3302′ can be coupled to the spinal rod 3260 at any distance from the connector body 3204 along the length of the spinal rod 3260.

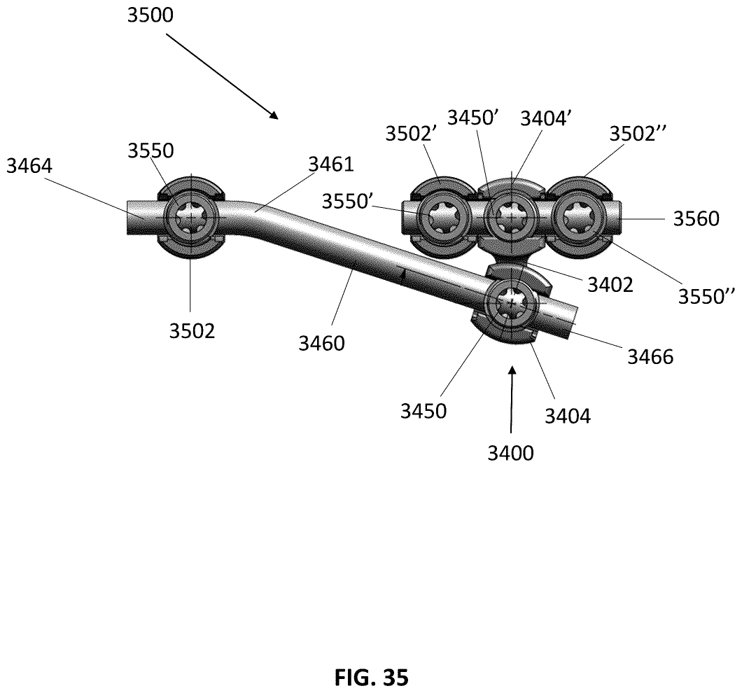

FIG. 35 illustrates another embodiment of a posterior fixation construct 3500 utilizing a modular connector assembly 3400 of the present disclosure. In FIG. 35, the modular connector assembly 3400 includes at least a first connector body 3404, a second connector body 3404′, and a connector rod 3402. This “U-U” connector can be similar to the connector shown in FIG. 17A in that at least one of the connector bodies 3404, 3404′ can be configured to pivot about the connector rod 3402. The first and second connector bodies 3404, 3404′ can include any of the features disclosed herein. The first connector body 3404 is coupled to a first spinal rod 3460 via a set screw 3450. The first spinal rod 3460 includes a curvature 3461 at a first end 3464 of the first spinal rod 3460. A first bone anchor 3502 is coupled to the first end 3464 of the first spinal rod 3460 via a set screw 3550, and is disposed a distance from the first connector body 3404, opposite the curvature 3461. The second connector body 3404′ is coupled to a second spinal rod 3560 via a set screw 3450′. The second connector body 3404′ is flanked on opposite sides by a second and third bone anchors 3502′, 3502″. Each of the second and third bone anchors 3502′, 3502″ are coupled via set screws 3550′, 3550″ to the second spinal rod 3560.

In this configuration, the connector rod 3402 is pivoted about the socket (not shown) of the first connector body 3404 such that a longitudinal axis of a second end 3466 of the first spinal rod 3460 extends obliquely to a longitudinal axis of the second spinal rod 3560. A longitudinal axis of the first end 3464 of the first spinal rod 3460 can be positioned parallel and in-line with the longitudinal axis of the second spinal rod 3560 due to the curvature 3461 at the first end 3464 of the first spinal rod 3460.

From this and the other illustrations included herein, it is apparent that the modular connector assemblies of the present disclosure can enable the creation of a variety of fixation constructs and provide a versatile tool to surgeons and other users. A wide variety of individual component pieces can be combined in various manners to create varied assemblies, all of which can utilize some common base components (e.g., set screws, rod seats, etc.). Furthermore, the modular connector assemblies of the present disclosure can be manufactured in any of a variety of sizes, such that they can be utilized with different sizes of spinal fixation rods or other fixation elements, bone screws, etc. For example, larger modular assemblies can be utilized in lower areas of the spine, such as the lumbar and sacrum regions, while smaller modular assemblies can be utilized in higher areas of the spine, such as the thoracic and cervical regions.

Various devices and methods disclosed herein can be used in minimally-invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, nickel, cobalt-chromium, or alloys and combinations thereof, polymers such as PEEK, ceramics, carbon fiber, and so forth. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A modular connector assembly comprising:
   a first connector having a first opening therein that is configured to receive a spinal fixation element;
   a second connector extending into the first connector through a second opening formed therein, the second connector having a bore formed in a first end thereof that is disposed within the first connector; and
   a coupler disposed within the first connector and passing through the bore of the second connector to prevent separation of the first connector and the second connector.
2. The assembly of example 1, further comprising a set screw threadably coupled to the first connector.
3. The assembly of example 2, further comprising a spinal fixation element disposed within the first opening of the first connector, wherein the set screw urges the spinal fixation element toward the coupler.
4. The assembly of any of examples 1 to 3,
   wherein a second end of the second connector extends through a first opening of a third connector; and
   wherein a second coupler is disposed within the third connector and passing through a bore formed in the second end of the second connector to prevent separation of the second connector and the third connector.
5. The assembly of example 4, wherein at least one of the first connector and the third connector is configured to pivot relative to the second connector.
6. The assembly of any of examples 1 to 5, wherein the first opening is substantially U-shaped with an open proximal end and is defined by spaced apart arms of the first connector.
7. The assembly of any of examples 1 to 5, wherein the first opening is formed through opposed sidewalls of the first connector with a closed proximal end.
8. The assembly of any of examples 1 to 7, wherein the first connector includes an inner threaded surface configured to interface outer threads of a set screw.
9. The assembly of any of examples 1 to 8, wherein the second opening of the first connector has a shape that substantially matches a shape of a portion of the second connector that extends therethrough.
10. The assembly of any of examples 1 to 8, wherein the second opening of the first connector has a shape that is larger in one dimension than a shape of a portion of the second connector that extends therethrough such that the second connector can pivot in one direction relative to the first connector.
11. The assembly of example 10, further comprising a spring disposed within the first connector and configured to impart a drag force on pivoting movement of the second connector relative to the first connector.
12. The assembly of any of examples 1 to 11, wherein the second connector comprises a rod extending from the first connector.
13. The assembly of example 12, wherein a second end of the rod has a flange.
14. The assembly of any of examples 1 to 13, wherein the second connector comprises spaced apart arms defining a substantially C-shaped opening with an open end opposite the first end of the second connector that is disposed within the first connector.
15. The assembly of any of examples 1 to 13, wherein the second connector comprises an opening formed through sidewalls thereof with a closed proximal end.
16. The assembly of any of examples 1 to 13, wherein the second connector comprises a hook formed opposite of the first end of the second connector that is disposed within the first connector.
17. The assembly of any of examples 1 to 13, wherein the second connector comprises a plurality of auxiliary fixation openings formed opposite of the first end of the second connector that is disposed within the first connector.
18. The assembly of any of examples 1 to 13, wherein the second connector comprises a rectangular body with a plurality of bores formed therein opposite the first end of the second connector that is disposed within the first connector.
19. The assembly of any of examples 1 to 18, wherein the coupler comprises a proximal-facing rod receiving surface.
20. The assembly of example 19, wherein the rod receiving surface comprises any of a curved surface and opposed planar surfaces that are angled toward one another.
21. The assembly of any of examples 1 to 20, wherein the coupler comprises a distally-extending post configured to extend through the bore formed in the second connector.
22. The assembly of any of examples 1 to 21, wherein the coupler comprises spaced apart arms configured to interface with a retention feature on an inner surface of the first connector to prevent removal of the coupler from the first connector.
23. A method of assembling a modular connector, comprising:
   inserting a first connector through an opening of a second connector to dispose a first end of the first connector within the second connector; and
   inserting a coupler through a second opening of the second connector and a bore formed in the first end of the first connector to prevent separation of the first connector and the second connector.
24. The method of example 23, further comprising threading a set screw into the first connector.
25. The method of example 24, further comprising inserting a spinal fixation element into the first connector and rotating the set screw to urge the spinal fixation element toward the coupler.
26. The method of any of examples 23 to 25, further comprising:
   inserting a second end of the first connector into an opening of a third connector; and
   inserting a second coupler into the third connector and through a bore formed in the second end of the first connector to prevent separation of the first connector and the third connector.
27. The method of example 26, further comprising pivoting at least one of the second connector and the third connector relative to the first connector.
28. The method of any of examples 23 to 27, further comprising inserting a spring into the second connector to impart a drag force on pivoting movement of the first connector relative to the second connector.
29. The method of example 28, further comprising inserting the coupler through a bore formed in the spring.
30. The method of any of examples 23 to 29, further comprising pivoting the first connector relative to the second connector.

What is claimed is:

1. A modular connector assembly comprising:
a first connector having a first opening therein that is configured to receive a spinal fixation element;
a second connector extending into the first connector through a second opening formed therein, the second connector having a bore formed in a first end thereof that is disposed within the first connector; and
a coupler disposed within the first connector and passing through the bore of the second connector to prevent separation of the first connector and the second connector.

2. The assembly of claim 1, further comprising a set screw threadably coupled to the first connector.

3. The assembly of claim 2, further comprising a spinal fixation element disposed within the first opening of the first connector, wherein the set screw urges the spinal fixation element toward the coupler.

4. The assembly of claim 1,
wherein a second end of the second connector extends through a first opening of a third connector; and
wherein a second coupler is disposed within the third connector and passing through a bore formed in the second end of the second connector to prevent separation of the second connector and the third connector.

5. The assembly of claim 4, wherein at least one of the first connector and the third connector is configured to pivot relative to the second connector.

6. The assembly of claim 1, wherein the first opening is substantially U-shaped with an open proximal end and is defined by spaced apart arms of the first connector.

7. The assembly of claim 1, wherein the first opening is formed through opposed sidewalls of the first connector with a closed proximal end.

8. The assembly of claim 1, wherein the first connector includes an inner threaded surface configured to interface outer threads of a set screw.

9. The assembly of claim 1, wherein the second opening of the first connector has a shape that substantially matches a shape of a portion of the second connector that extends therethrough.

10. The assembly of claim 1, wherein the second opening of the first connector has a shape that is larger in one dimension than a shape of a portion of the second connector that extends therethrough such that the second connector can pivot in one direction relative to the first connector.

11. The assembly of claim 10, further comprising a spring disposed within the first connector and configured to impart a drag force on pivoting movement of the second connector relative to the first connector.

12. The assembly of claim 1, wherein the second connector comprises a rod extending from the first connector.

13. The assembly of claim 12, wherein a second end of the rod has a flange.

14. The assembly of claim 1, wherein the second connector comprises spaced apart arms defining a substantially C-shaped opening with an open end opposite the first end of the second connector that is disposed within the first connector.

15. The assembly of claim 1, wherein the second connector comprises an opening formed through sidewalls thereof with a closed proximal end.

16. The assembly of claim 1, wherein the second connector comprises a hook formed opposite of the first end of the second connector that is disposed within the first connector.

17. The assembly of claim 1, wherein the second connector comprises a plurality of auxiliary fixation openings formed opposite of the first end of the second connector that is disposed within the first connector.

18. The assembly of claim 1, wherein the second connector comprises a rectangular body with a plurality of bores formed therein opposite the first end of the second connector that is disposed within the first connector.

19. The assembly of claim 1, wherein the coupler comprises a proximal-facing rod receiving surface.

20. The assembly of claim 19, wherein the rod receiving surface comprises any of a curved surface and opposed planar surfaces that are angled toward one another.

21. The assembly of claim 1, wherein the coupler comprises a distally-extending post configured to extend through the bore formed in the second connector.

22. The assembly of claim 1, wherein the coupler comprises spaced apart arms configured to interface with a retention feature on an inner surface of the first connector to prevent removal of the coupler from the first connector.
